(19) United States
(12) Patent Application Publication
(10) Pub. No.: US 2024/0035059 A1
(43) Pub. Date: Feb. 1, 2024

(54) A HIGHLY SENSITIVE HEAT-REPRESSIBLE SPLIT-T7 POLYMERASE (THERMAL-T7RNAP) FOR APPLICATIONS IN BIOTECHNOLOGY

(71) Applicant: National University of Singapore, Singapore (SG)

(72) Inventors: Wai Kit Chee, Singapore (SG); Jing Wui Yeoh, Singapore (SG); Viet Linh Dao, Singapore (SG); Chueh Loo Poh, Singapore (SG)

(73) Assignee: National University of Singapore, Singapore (SG)

(21) Appl. No.: 18/255,645

(22) PCT Filed: Dec. 1, 2021
(86) PCT No.: PCT/SG2021/050744

§ 371 Date: Jun. 2, 2023

(30) Foreign Application Priority Data
Dec. 2, 2020 (SG) 10202012000X

(51) Int. Cl. C12P 19/34; C12N 15/70; C12N 9/12

(52) U.S. Cl. CPC C12P 19/34; C12N 15/70; C12N 9/1247; C12Y 207/07006; C12N 2800/101

(57) ABSTRACT

The present invention relates to heat-repressible split-T7 polymerases comprising temperature-sensitive domains of Tlpa protein fused with split T7 RNA polymerase (T7RNAP) to introduce thermal control into widely used T7 RNA polymerase, creating a heat-repressible Thermal-T7RNAP system. The invention further provides polynucleotides encoding the heat-repressible split-T7 polymerases, and methods of thermal control of bioproduction.

### FIELD OF THE INVENTION

The present invention relates to heat-repressible split-T7 polymerases comprising temperature-sensitive domains of Tlpa protein fused with split T7 RNA polymerase (T7RNAP) to introduce thermal control into widely used T7 RNA polymerase, creating a heat-repressible Thermal-T7RNAP system. The invention further provides polynucleotides encoding the heat-repressible split-T7 polymerases, and methods of thermal control of bioproduction.

### BACKGROUND OF THE INVENTION

Existing heat-repressible systems either function at too low temperatures (<30° C.) for optimal enzymatic bioprocesses to occur, suffer from low fold inductions and long delays or lack orthogonal control.

The heat-repressible systems generally fall into two main categories, RNA based or transcriptional regulations. RNA based thermometers typically suffer from long delay in responses and small fold inductions (<10-fold) [Hoynes-O'Connor, A. et al., Nucleic Acids Res 43:6166-6179 (2015)]. In the second category, there are very limited heat-repressible systems based solely on transcriptional regulation. The closest example was the coupling of an inverter with the heat-inducible transcription repressor cl434 to enable a low temperature input (25° C.) be converted into high expressions [Yang, Z. et al., Nucleic Acids Research 47(21):e137 (2019)]. By introducing additional complexity into the thermal system, it is likely to result in the circuit being intricately tied to the host's metabolism and become unstable as it is hyper-sensitized to global cellular effects [Segall-Shapiro, T. H. et al., Mol Syst Biol 10:742 (2014)].

To address these issues, a compact and orthogonal thermal system is of great significance to minimize the burden to the host. Among the numerous phage-based polymerases, the T7 RNA polymerase (T7RNAP) is a popular variant of orthogonal RNAP in the field of biotechnology [Wang, W. et al., Biotechnol Adv. 36:2129-2137 (2018)]. The polymerase can effectively decouple transcription from the host and recognize specifically its cognate T7 promoter [Segall-Shapiro, T. H. et al., Mol Syst Biol 10:742 (2014)]. These advantages have been recognized early and led to efforts in making T7RNAP system thermal controllable which mainly involved placing the T7RNAP gene under heat-inducible operons [Wang, Z. W. et al., Biotechnology Progress 20:1352-1358 (2004); Chao, Y. P. et al., Appl Microbiol Biotechnol 58:446-453 (2002)]. Fundamentally many protein interactions are modular in nature and it is a common strategy for native proteins to be fused with foreign domains, to convert a physical or chemical signal into a change in physical interactions between the fusion pairs [Grunber, T. and Serrano, L. Nucleic Acids Res 38:2663-2675 (2010)]. The fusion of light sensitive protein domains with the split N-terminal and C-terminal T7RNAP fragments represents the recent work in providing a means of using physical cue to control the activity of the polymerase directly, [Han, T. et al. ACS Synth Biol 6:357-366 (2017); Baumschlager, A. et al., ACS Synth Biol 6:2157-2167 (2017)]. Along the same vein, it is likely to introduce direct thermal control into T7RNAP by its fusion with temperature-sensitive domains. An early attempt to introduce temperature-sensitive intein into T7RNAP gene [Liang, R. et al., J Microbiol Methods 68:497-506 (2007)] was compounded with a number of limitations. Firstly, the system was activated at a low temperature of 18° C., which makes many bioprocesses non-optimal. Secondly, temperature-mediated splicing is slow and takes several hours [Shah, N. H. and Muir, T. W., Chem Sci 5:446-461 (2014)]. Thirdly, the splicing requires extensive design before achieving optimum activity. Lastly, the ‘permanent nature’ of this post-translational modification has limited its applicability.

There is a need for improved methods and constructs for heat-repressible production of recombinant proteins or small molecules.

### SUMMARY OF THE INVENTION

While many studies reported on the development of heat-inducible systems [Piraner, D. I. et al., Nat Chem Biol 13:75-80 (2017); Rodrigues, J. L. and Rodrigues, L. R. Trends Biotechnol 36:186-198 (2018)], very few heat-repressible systems have been developed. Nevertheless, low temperature in general remains useful for improving protein solubility, increasing its stability by promoting proper folding and reduce the detrimental effects from toxic stress proteins [Qing, G. et al., Nat Biotechnol 22:887-882 (2004); Sorensen, H. O and Mortensen, K. K., Microb Cell Fact 4:1 (2005)]. However, earlier reported systems switch on at very low temperature (e.g., <30° C.). The milder temperatures can also lead to more efficient bio-catalysis processes and increases the yield of unstable heat-labile proteins [Singh, R. et al., 3 Biotech 6174-174 (2016)]. Known heat-inducible systems and their characteristics are listed in Table 1, in comparison to the heat-repressible thermal T7RNAP system of the invention.

TABLE 1
Heat-repressible thermal sensors presented in literature. * Indicates the best specification.

| Thermal | Activation | Sensitive | Fold- | | | | |
|---|---|---|---|---|---|---|---|
| biosensor | temp. | range | induction | Response | Reversbility | Orthogonality | Ref. |
| cspA mRNA | 15° C. | 15-37° C. | 500-fold* | No data | No data | No | * |
| | | | | (but | | | |
| | | | | protein | | | |
| | | | | expression | | | |
| | | | | after 12 | | | |
| | | | | hours | | | |
| RNA | 27° C. | 27-37° C. | <10-fold | 40-70 | No data | No | \hat{ } |
| thermometer | | | | mins | | | |
| (RNAse E) | | | | | | | |
| cl434 | 25° C. | 25-40° C. | 84-fold | Within | No data | No | % |
| (inverted | | | | hours | | | |
| circuit) | | | | | | | |
| T7RNAP | 18° C. | 18-37° C. | 7-fold | No data | No data | Yes, using | ~ |
| disrupted by | | | | | | T7RNAP | |
| intein | | | | | | | |
| Thermal- | 30° C. | 30-40° C. | 31-fold | Within | Yes, highly | Yes using | This |
| T7RNAP | | (narrow)* | | minutes* | dynamics* | TYRNAP* | work |

*Qing et al., Nat Biotechnol 22: 877-882 (2004).
\hat{ } Hoynes-O'Connor, A., et al., Nucleic Acids Res 43, 6166-6179 (2015).
% Yang Zheng, et al., Nucleic Acids Research 47, e137. 10.1093/nar/gkz785 (2019).
~ Liang, R., et al., J Microbiol Methods 68, 497-506 (2007).

Accordingly, an alternative mode of thermal regulation was developed by providing a reversible and tunable thermal-repressible split-T7 RNA polymerase systems (Thermal-T7RNAPs) which fuses temperature-sensitive domains of Tlpa protein with split-T7RNAP to enable direct thermal control of the T7RNAP activity between 30-42° C.

According to a first aspect of the invention, there is provided an isolated heat-repressible Split-T7 polymerase fusion protein, comprising:
(i) a split T7 RNA polymerase polypeptide (T7RNAP);
(ii) a polypeptide coiled-coil domain; and
(iii) a linker peptide between the polypeptides (i) and (ii),
   wherein an N-terminal fragment of T7 RNA polymerase (T7RNAP) is fused to a polypeptide coiled-coil domain and a C-terminal fragment of T7 RNA polymerase is fused to a polypeptide coiled-coil domain.

In some embodiments, the coiled-coil domain is selected from the group comprising TlpA polypeptide, M class C proteins from group A streptococci, such as Arp4 and Sir22, and Hv1/VSOP voltage-gated H⁺ channel protein.

In some embodiments, the N-terminal fragment of T7 RNA polymerase and the C-terminal fragment of T7 RNA polymerase are derived by splitting the T7 RNA polymerase polypeptide at amino acid position 563/564 of the mature peptide sequence.

In some embodiments, the T7RNAP comprises an amino acid sequence with at least 90% sequence identity with the amino acid sequence is set forth in SEQ ID NO: 1 and/or the TlpA coiled-coil comprises an amino acid sequence with at least 90% sequence identity with the amino acid sequence set forth in SEQ ID NO: 3.

In some embodiments, the coiled-coil domain fused to the C-terminal fragment of T7 RNA polymerase comprises one or more domains, X1, X2, X3, X4 and X5, encoded by polynucleotide sequences having at least 70%, at least 80%, at least 90% or 100% identity with sequences selected from the group comprising X1 (SEQ ID NO: 14), X2 (SEQ ID NO: 15), X3 (SEQ ID NO: 16), X4 (SEQ ID NO: 17) and X5 (SEQ ID NO: 18) or combinations thereof.

In some embodiments, the coiled-coil domain fused to the C-terminal fragment of T7 RNA polymerase comprises a X1 domain at the N-terminal end and a X4 domain at the C-terminal end of the coiled-coil domain.

In some embodiments, the polynucleotide sequence set forth in X1 (SEQ ID NO: 14) comprises a G/A substitution at position 52 and/or the polynucleotide sequence set forth in X4 (SEQ ID NO: 17) comprises a T/A substitution at position 4.

In some embodiments, the coiled-coil domain fused to the C-terminal fragment of T7 RNA polymerase comprises a plurality of X5 domains.

In some embodiments, the coiled-coil domain fused to the C-terminal fragment of T7 RNA polymerase comprises, from N-terminal to C-terminal, domains X1, X5, X5 and either X4 or X5. Preferably, the coiled-coil domain fused to the C-terminal fragment of T7 RNA polymerase is encoded by a polynucleotide sequence comprising the sequence set forth in SEQ ID NO: 12.

In some embodiments, the coiled-coil domain fused to the C-terminal fragment of T7 RNA polymerase consists of, from N-terminal to C-terminal, domains X1, X2, X3 and either X4 or X5. Preferably, the coiled-coil domain fused to the C-terminal fragment of T7 RNA polymerase is encoded by a polynucleotide sequence comprising the sequence set forth in SEQ ID NO: 13.

In some embodiments, as a result of varying combinations of coiled-coil domains X1, X2, X3, X4 and/or X5, the active temperature range can be tuned.

In some embodiments, the Split-T7 polymerase is active at temperatures in the range of about 30° C. to about 39° C. and is thermally repressed above 39° C.; preferably repressed at 37° C. and above.

According to a second aspect of the invention, there is provided an isolated nucleic acid molecule capable of expressing the fusion protein of any embodiment of the first aspect.

According to a third aspect of the invention, there is provided a plasmid or vector comprising the nucleic acid molecule of the second aspect.

According to a fourth aspect of the invention, there is provided a host cell comprising the nucleic acid molecule of the second aspect and/or the plasmid or vector of the third aspect, and a gene encoding a product of interest operably linked to a T7 promoter.

According to a fifth aspect of the invention, there is provided a composition comprising the host cell of the fourth aspect.

According to a fifth aspect of the invention, there is provided a method of regulating the relative proportions of two or more cell populations within a co-culture, comprising:
i) engineering a first cell to comprise the nucleic acid molecule of the second aspect and/or the plasmid or vector of the third aspect, and a growth regulatory gene operably linked to a T7 promoter, wherein said first cell comprises a Split-T7 polymerase which is active within a first temperature range;
ii) engineering a second cell to comprise a growth regulatory gene operably linked to a heat-inducible promoter; and/or
iii) engineering further cells to comprise the nucleic acid molecule of the second aspect and/or the plasmid or vector of the third aspect, and a growth regulatory gene operably linked to a T7 promoter, wherein each of said further cells comprise a Split-T7 polymerase which is active within a fully overlapping, a partially overlapping or non-overlapping temperature range to that of said first cell and/or each other,
   wherein raising or lowering the temperature of the co-culture regulates the growth of the respective first, second and/or further cell populations.

In some embodiments, the respective growth regulatory genes are the same.

In some embodiments, the respective growth regulatory genes slow down cellular growth, such as by limiting glucose uptake by expressing a SgrS sRNA which functions to degrade ptsG mRNA that encodes for a glucose transporter, IICB^{Glc}.

According to a sixth aspect of the invention, there is provided a kit comprising:
(i) the nucleic acid molecule of the third aspect and/or the plasmid or vector of the fourth aspect; and
(ii) a reaction buffer.

In some embodiments, the kit further comprises one or more ribonucleoside triphosphates.

According to a seventh aspect of the invention, there is provided a method of synthesizing an RNA molecule comprising:
(a) combining an isolated nucleic acid molecule of the second aspect and/or the plasmid or vector of the third aspect, with ribonucleoside triphosphates and/or a modified nucleotide and a template DNA molecule comprising a T7 RNA polymerase promoter that is operably linked to a target nucleotide sequence to be transcribed, to produce a reaction mix; and
(b) incubating the reaction mix to transcribe the template DNA molecule into RNA.

In some embodiments, the incubating is done at a temperature of less than 40° C., such as in a range of 30° C. to 39° C., preferably 37° C. or less, such as in a range of 30° C. to 37° C.

### BRIEF DESCRIPTION OF THE FIGURES

FIG. 1A-B shows (A) the original Tlpa repressor protein comprising a DNA-binding domain and a coiled-coil domain and (B) a Thermal-T7RNAP system wherein the DNA-binding domain of Tlpa is replaced by an N-terminal fragment of T7 protein and a separate coiled-coil domain absent the DNA-binding domain has a C-terminal fragment of TT protein fused at its C-terminal end.

FIG. 2a-g shows the principal behind direct thermal control of Thermal-T7RNAP. (a) The NT7 and CT7 protein fragments are fused to the temperature-sensitive coiled-coil domain of the Tlpa protein, via a short linker. The coiled-coil domain consists of the longer coiled-coil (94-257^{th}) and the shorter coiled-coil (258-371^{th}) regions. (b) At high temperatures, the Tlpa coiled-coil undergoes temperature-dependent uncoiling and allow sequestering of the NT7 and CT7 domains, thus rendering the polymerase inactive for downstream expression to occur. At low temperature, the coiled-coil retains a dimer complex, allowing the NT7 and CT7 domains to be reconstituted into an active polymerase and capable of driving downstream gene expression. (c) After an 18-hour incubation period in the thermal regulation assay, the corresponding experimental data (GFP/OD₆₀₀) (filled circles) were normalised to the lowest values at 42° C. and plotted. The dose responses were represented by the Hill equation which were indicated as solid lines. The Hill coefficient, n and half-activation temperature, Kₘ, are indicated for some systems (Table 2). (d) Corresponding fluorescence (GFP/OD₆₀₀) levels after 18 hours. Constitutive promoters of varying strengths were used in control of the expression levels of NT7-Tlpa coil protein, while the CT7-Tlpa coil protein was being expressed at a constant level using J23101 promoter on another plasmid of similar copy number. An alternative gene configuration B was also adopted to express both proteins on the same plasmid under the strong J23101 promoter. The genetic circuits are shown in FIG. 3. The Tlpa* system represented the fusion of the individual NT7 and CT7 fragments with truncated coiled-coil domain through removal of the shorter coiled-coil (258-371^{th} residues). (e) Predicted fluorescence expression for the Thermal-T7RNAPs after 18 hours. (f) The predicted amount of unbounded NT7 and CT7 fusion proteins at 30° C. (g) The corresponding predicted amounts of reconstituted T7RNAP proteins at 30° C. and 42° C. Data information: The experimental data are represented as mean±S.D. (n=3). Statistical significances of ***P<0.001 and **P<0.01 were calculated based on two-sample unpaired t-test. The corresponding fold-change between the temperatures or the split proteins were shown above each of the bar charts.

FIG. 3a-b shows genetic circuits of Thermal-T7RNAP systems. (a) Gene configuration A. In Thermal-T7RNAP(v1) and Thermal-T7RNAP(v2) respectively, constitutive promoters of varying strengths (gabDP2-‘low’ and J23101-‘high’) are used in control of the expression levels of NT7-Tlpa coil protein, while the CT7-Tlpa coil protein is being expressed at a constant level using J23101 promoter on another plasmid of similar copy number. (b) Gene configuration B. In Thermal-T7RNAP(v3), the NT7-Tlpa coil and CT7-Tlpa coil are expressed on the same plasmid under J23101 promoters.

FIG. 4a-e shows thermal control of vanillin bioproduction. (a) Genetic circuit for vanillin bio-conversion. Guided by the parental backbone Thermal-T7RNAP(v3) which contained the gene configuration B the two enzymes, fcs and ech, are expressed as an operon under the control of the pT7 promoter. (b) Vanillin bio-conversion pathway. Ferulic acid is bio-transformed into vanillin using the two enzymes in a multi-step pathway. The experimental timeline is shown below. (c) The vanillin product concentrations. The concentrations were calculated by normalizing the actual concentration (%) to the dry cell weight (DCW). Corresponding ferulic acid and vanillin concentrations of the Full length T7-WT control was also depicted in the smaller figure. (d) Percentage yield of vanillin. The vanillin yields were calculated by taking the percentage ratio between experimentally measured yields and the theoretical yield of vanillin (0.078%). (e) Vanillin productivity. The productivity was computed by taking the difference between final vanillin concentration and initial vanillin concentration (0%) and normalized to the time required for complete bio-conversion to occur (5 hrs).

FIG. 5a-h shows dynamic control of Thermal-T7RNAPs. (a) Fluorescence expression profile for Thermal-T7RNAP(v1) and Thermal-T7RNAP(v3) under patterned 3-hour (h)-3 h-2 h-2 h OFF-ON thermal cycle, when alternating between 40° C. and 30° C. Measured temperature profiles over the 10-hour period are indicated. Values were normalised to the maximal GFP expression levels. (b) Corresponding GFP synthesis rates are shown. Absolute fluorescence expression profiles of both systems under (c) patterned 3 h-3 h-2 h-2 h thermal cycle and (d) single-step 3 h-7 h, when alternating between 37° C. and 30° C. The absolute values enable ease of comparison of maximum expression levels between systems across the different time interval patterns. Fluorescence expression profiles for Thermal-T7RNAP(v3) under constant (e) 3 h and (f) 2 h intervals, when alternating between 37° C. and 30° C., over 12-hour duration. The experiment data are shown as filled circles. The ‘Prediction’ line represented the predicted profiles derived from a constant temperature regime. The ‘Refitted’ line represented the improved predicted profiles after model-refitting and adjustment in model parameters. Corresponding GFP synthesis rates of Thermal-T7RNAP(v3) under constant (g) 3 h and (h) 2 h intervals are also shown. The ‘Prediction 1’ line represented the predicted profiles derived from a constant temperature regime. The ‘Prediction 2’ line represented the improved predicted profiles after model-refitting and adjustment in model parameters. Data information: the experimental data are represented as mean±S.D. (n=3).

FIG. 6a-b shows the mutagenesis framework. (a) Genetic circuits of Thermal-T7RNAP(v1) system where the coiled-coil domains of the NT7 and CT7 protein fragments are mutated (indicated by the primer arrows). (b) Replicated plates after overnight incubation at the respective temperature. The individual colony are each picked and plated by colony picker. The highlighted colonies indicate shortlisted colonies with ideal thermal characteristics (‘ON’ states at low temperature).

FIG. 7a-g shows how automated screening of Thermal-T7RNAP mutants was performed. (a) Mutagenesis framework. In the first phase of the screening, (i) error-prone PCR was performed on the temperature-sensitive coiled-coil domains of the NT7 and CT7 protein fragments. Through this fusion protein assembly strategy, a pooled library of mutant fusion polymerases was created. The pooled library was subsequently screened in later steps. (ii) The mutant libraries were subsequently transformed and plated on agar plates. The individual overnight colonies were machine ‘picked’ by the colony picker and replicative plated on three identical agar plates for incubation at the desired ‘ON’ and ‘OFF’ state temperatures. (iii) Information of fluorescence and size of each colony was captured by the colony picker (iv-vi) within the agar plates at various temperature. The fold differences in fluorescence between (vii) 30° C. and 37° C. and (viii) 30° C. and 40° C. were computed to sort and identify potential mutants that exhibited the desired thermal characteristics. As an example, three potential candidates were highlighted. In the second phase, a high precision screening was conducted. (ix) Liquid characterization of the shortlisted colonies was conducted in the thermal regulation assay. (x) Each of the dose response was automatically fitted with Hill equation to extract important characteristics. (b-d) Thermal characteristics of the mutants. Two discovered mutants (Mut 1 and Mut 2) with distinctively different thermal activation ranges were highlighted. The Temperature_{10%} and Temperature_{90%} represent the temperatures at which the fluorescence levels are at 10% and 90% of their maximal fluorescence expressions respectively. (e) Mechanisms for the heat-repressible Thermal-T7RNAP system (top) and heat-inducible Tlpa system (bottom). At low temperature (30° C.), the coiled-coil domain of the Tlpa repressor protein maintained as a dimeric complex which allows the binding of the DNA binding domain to its promoter pTlpa to inhibit downstream repression. At high temperatures (>37° C.), the coiled-coil domains undergo temperature-dependent uncoiling which render the Tlpa repressor inactive, thus enabling of constitutive expression of the promoter. (f) Thermal dose responses after 18 hours incubation. (g) ‘Traffic light’ pattern. For the co-cultures, the respective cells were grown at equal initial proportions and plated on agar overnight to observe thermal logic. Data information: The experimental data are represented as mean±S.D. (n=3).

FIG. 8 shows the consensus domains (X1-X5) discovered. The nucleotide positions are all referenced to the positions within the original Tlpa protein. For X1 and X4 domains, the percentages represent the 98% homology to the wild type protein, while the remaining domains have 100% homology to the wild type protein.

FIG. 9A-C show sequence comparisons of Tlpa-conserved domains in Mut1 and Mut2 mutants; (A) Tlpa and Mut1; (B) Tlpa and Mut2; and (C) Mut1 and Mut2.

FIG. 10a-m shows thermal control of co-culture distribution. (a) Thermal modulation. At different temperatures, the cell proportions are regulated. (b) Growth inhibition mechanisms. The GFP reporting cells contained the heat-repressible Thermal-T7RNAP system and the RFP reporting cells contained the heat-inducible Tlpa system. Both systems regulate their cell growth by limiting glucose uptake at 30° C. and 40° C. respectively. (c,d) Final growth values for the monocultures (100%) at 30° C. and 40° C. after 8 hours. The GFP (G) and RFP (R) cells which contained the growth inhibition modules and their respective controls (no SgrS expression) were shown. (e,f) Combined growth curves for the co-cultures. The cells were initially seeded at equal proportion (G50%+R50%). The control co-culture does not express SgrS. Predicted growth curve of the co-culture (solid lines) and the experimental data (filled circles) were shown. (g) Final cell distributions in the co-culture. After 8 hours, the final compositions between the GFP population and RFP population were derived from their measured fluorescence levels (solid bars). The amount of GFP cells was computed as a percentage of the fluorescence intensities of the monoculture at 40° C. (repressed). The amount of RFP cells was obtained after subtraction from the GFP cells. The shaded bars represented the predicted cell distributions as derived from the ratio between individual ODs. (h) Change in OD ratios over time. The cells were initially seeded at equal proportion. (i) Combined growth curves under various initial cell proportions. Predicted growth curve of the co-culture (solid lines) and the experimental data (filled circles) were shown. (j,k) Predicted growth profiles of individual GFP and RFP. (l) Final OD ratios of the co-cultures. (m) Contour representation of initial and final cell proportions within the co-culture, under different temperatures. The cell proportions are obtained by dividing the predicted OD values of GFP cells with the RFP cells. The cross marks represented conditions performed in experiment. The highlighted blue region represented the conditions that maintained cell proportions in the 8-hours period. Data information: The experimental data are represented as mean±S.D. (n=3). Statistical significances of ***P<0.001 were calculated based on two-sample unpaired t-test. The fold change between the temperatures were indicated above each of the bar charts.

FIG. 11 shows genetic circuits for GFP_{ThermalT7RNAP-SgrS} and RFP_{Tlpa-SgrS} cells that will make up the co-culture. In the control co-culture, while maintaining similar genetic configuration, the ‘p_{T7}-rbs34-SgrS’ or ‘p_{Tlpa}-rbs34-SgrS’ cassette is removed.

FIG. 12a-j shows thermal modulation of co-culture with Thermal-T7RNAP mutants. (a,b) Continuous fluorescence intensities profiles of the co-culture library at 30° C. and 37° C. The GFP_{ThermalT7RNAP-SgrS(unmutated)}, GFP_{ThermalT7RNAP-SgrS(Mut1)} and GFP_{ThermalT7RNAP-SgrS(Mut2)} cells were each grown together with the RFP_{Tlpa-SgrS} cells at equal initial proportion to form the original, Mut 1 and Mut 2 co-culture systems respectively. (c) Corresponding fluorescence values after 250 mins. (d) Combined growth curves of the co-cultures. The experimental (filled circles) and predicted values (solid lines) were shown. (e) Predicted OD growth curve for the GFP population. (f) OD ratio profiles over time. The ratios were computed after diving the predicted OD values of the GFP population by the RFP population. The small arrow indicated data overlap between Original, Mut 1 and Mut 2 at 30° C. (g) Predicted OD values for GFP cells and (h) final OD ratios after 250 mins. (i,j) Change in OD ratios between initial and final conditions of the co-cultures under different initial cell proportions after 250 mins. Data information: The experimental data are represented as mean±S.D. (n=3). Statistical significances of ***P<0.001, **P<0.01 and *P<0.05 were calculated based on two-sample unpaired t-test. The corresponding fold-change between the temperatures were shown above each of the bar charts.

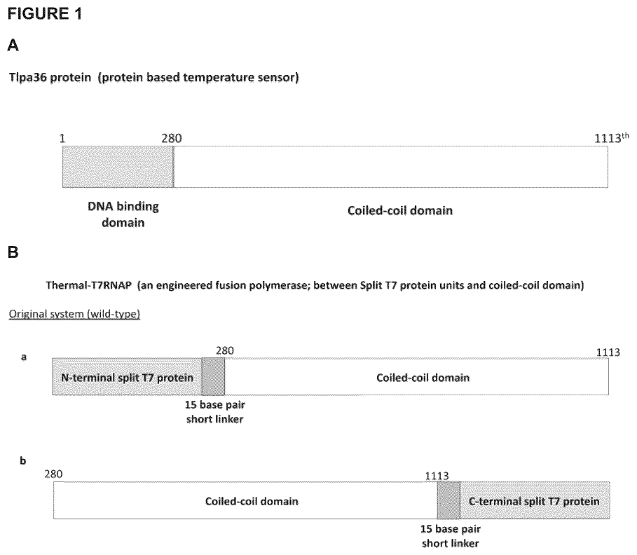

### DETAILED DESCRIPTION OF THE INVENTION

### Definitions

Certain terms employed in the specification, examples and appended claims are collected here for convenience.

As used in the specification and the appended claims, the singular forms “a,” “an” and “the” include plural referents unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Bibliographic references mentioned in the present specification are for convenience listed at the end of the examples. The whole content of such bibliographic references is herein incorporated by reference.

### EXAMPLES

### Example 1

### Plasmid Design and Construction

All plasmids were designed in silico using Benchling (Benchling, Inc. San Francisco, CA, USA). Individual gene fragments and primers were synthesized from Integrated DNA Technologies (Integrated Device Technology, Inc. San Rose, CA, USA). The polymerase chain reaction (PCR) products were amplified using Q5 High-Fidelity DNA polymerase (New England Biolabs, MA, USA) with strict accordance to the manufacturer's protocols. PCR products were analysed by gel electrophoresis using 1% agarose gel and purified using QIAquick gel extraction kit (Qiagen, Hilden, Germany). The DNA concentrations of the gel-purified samples were quantified with Nanodrop™ One^{c} (Thermo Fisher Scientific, MA, USA). Gibson assembly was subsequently performed using the NEBuilder HiFi DNA assembly (New England Biolabs, MA, USA) with strict accordance to the manufacturer's protocols. Subsequent assembly products were chemically transformed into E. coli K-12 strain NEB DH-10 Beta (New England Biolabs, MA, USA) unless stated otherwise. Colonies that grown on the LB-antibiotic plate were picked and inoculated into fresh LB-antibiotic medium at 37° C. to prepare overnight culture for plasmids extraction using QIAprep Spin Miniprep kit (Qiagen, Hilden, Germany). The plasmids were then sent for DNA sequencing (1st BASE, Singapore). The sequencing results were subsequently aligned with the digital template and analysed on Benchling platform.

pBbE6K (JBEI Part ID: JPUB 000054, colE1 ori, Kan^{r}), pBbE8K (JBEI Part ID: JPUB 000036, colE1ori, Kan^{r}), pBbA6C (JBEI Part ID: JPUB 000056, p15A ori, Cm^{r}), pBbA8C (JBEI Part ID: JPUB 000038, p15A ori, Cm^{r}) and pNO4, which was a gift from Jeffrey Tabor (pSC101 ori, Amp^{r}) (Addgene plasmid #101066; n2tdotnet/addgene:101066; RRID:Addgene_101066), were used as the backbones in the plasmid constructions whenever necessary. Constitutive promoters J23101 (BBa_J23101) and gabDP2 (BBa_K3252022), pT7 promoter (BBa_R0085), double terminator 15T (BBa_B0015), rrnBT1 terminator (BBa_B0010), T7 terminator (BBa_K731721), ribosome binding site (rbs34) (BBa_B0034), green fluorescence protein gene GFPmut3b (BBa_E0040), red fluorescence protein gene DsRed (BBa_K2782004), sugar transport related sRNA gene (SgrS) (BBa_K581005) and the T7 RNA polymerase gene (BBa_12032) were obtained from iGEM Registry of Standard Biological Parts (iGEM Foundation, Cambridge, MA, USA) (http://partsregistry.org) and used in plasmid constructions whenever necessary. The T7 RNA polymerase gene was split at the 563 (S)/564^{th} (E) location, to form the N-terminal T7 protein unit and C-terminal T7 protein unit, as guided by previous work when attempting to find the most optimum split sites [Baumschlager et al., ACS Synth Biol 6:2157-2167 (2017); Han et al., ACS Synth Biol 6:357-366 (2017)]. The GGSGG linker was obtained from a previous study (Baumschlager et al., ACS Synth Biol 6:2157-216 (2017)]. The full length 371 amino acids (a.a) long Tlpa36 gene (referred herein as Tlpa) and its pTlpa promoter were derived from the paper (Piraner et al., Nat Chem Biol 13:75-80 (2017)], and synthesised as a gene fragment. The list of promoters, RBS and relevant gene sequences are listed in Table 2.

TABLE 2
Amino acid and DNA sequences of genetic elements used in the study.

| Genetic | | |
|---|---|---|
| Element | Description | Amino acid/nucleotide sequence |
| T7RNAP | Wild type | MNTINIAKNDFSDIELAAIPFNTLADHYGERLAREQLALEHESYEMG |
| SEQ ID | (split site | EARFRKMFERQLKAGEVADNAAAKPLITTLLPKMIARINDWFEEVK |
| NO: 1 | highlighted) | AKRGKRPTAFQFLQEIKPEAVAYITIKTTLACLTSADNTTVQAVASAI |
| | | GRAIEDEARFGRIRDLEAKHFKKNVEEQLNKRVGHVYKKAFMQVV |
| | | EADMLSKGLLGGEAWSSWHKEDSIHVGVRCIEMLIESTGMVSLHR |
| | | QNAGVVGQDSETIELAPEYAEAIATRAGALAGISPMFQPCVVPPKP |
| | | WTGITGGGYWANGRRPLALVRTHSKKALMRYEDVYMPEVYKAINI |
| | | AQNTAWKINKKVLAVANVITKWKHCPVEDIPAIEREELPMKPEDID |
| | | MNPEALTAWKRAAAAVYRKDKARKSRRISLEFMLEQANKFANHKA |
| | | IWFPYNMDWRGRVYAVSMFNPQGNDMTKGLLTLAKGKPIGKEGY |
| | | YWLKIHGANCAGVDKVPFPERIKFIEENHENIMACAKSPLENTWW |
| | | AEQDSPFCFLAFCFEYAGVQHHGLSYNCSLPLAFDGSCSGIQHFS |
| | | AMLRDEVGGRAVNLLPSETVQDIYGIVAKKVNEILQADAINGTDNE |
| | | VVTVTDENTGEISEKVKLGTKALAGQWLAYGVTRSVTKRSVMTLA |
| | | YGSKEFGFRQQVLEDTIQPAIDSGKGLMFTQPNQAAGYMAKLIWE |
| | | SVSVTVVAAVEAMNWLKSAAKLLAAEVKDKKTGEILRKRCAVHWV |
| | | TPDGFPVWQEYKKPIQTRLNLMFLGQFRLQPTINTNKDSEIDAHKQ |
| | | ESGIAPNFVHSQDGSHLRKTVVWAHEKYGIESFALIHDSFGTIPAD |
| | | AANLFKAVRETMVDTYESCDVLADFYDQFADQLHESQLDKMPALP |
| | | AKGNLNLRDILESDFAFA...883 |
| Tlpa | Coding | MRPATYEPEQIIEAGLALQAEGRNITGFALRNQVGGGNPTRLRQIW |
| (Tlpa36) | sequence | DEYQASQSTVVTELVAELPVEVAEEVKAVSAALSERITQLATELND |
| SEQ ID | (coiled coil | KAVRAAERRVAEVTRAAGEQTAQAERELADAAQTVDDLEEKLVEL |
| NO: 2 | domain | QDRYDSLTLALESERSLRQQHDVEMAQLKERLAAAEENTRQREE |
| | highlighted) | RYQEQRTVLQDALNAEQAQHINTREDQQKRLEQISAEANARTEEL |
| | | KSERDKVNTLLTRLESQENALASERQQHLATRETLQQRLEQAIADT |
| | | QARAGEIALERDRVSSLTARLESQEKASSEQLVRMGSEIASLTERC |
| | | TQLENQRDDARLETMGEKETVAALRGEAEALKRQNQSLMAALSG |
| | | NKQTGGQNA |
| Tlpa | coiled coil | AVRAAERRVAEVTRAAGEQTAQAERELADAAQTVDDLEEKLVELQ |
| (Tlpa36) | domain | DRYDSLTLALESERSLRQQHDVEMAQLKERLAAAEENTRQREER |
| SEQ ID | | YQEQRTVLQDALNAEQAQHINTREDQQKRLEQISAEANARTEEL |
| NO: 3 | | KSERDKVNTLLTRLESQENALASERQQHLATRETLQQRLEQAIAD |
| | | TQARAGEIALERDRVSSLTARLESQEKASSEQLVRMGSEIASLTER |
| | | CTQLENQRDDARLETMGEKETVAALRGEAEALKRQNQSLMAA |
| | | LSGNKQTGGQNA...278 |
| PT7 | Unregulated | TAATACGACTCACTATAGGG |
| SEQ ID | pT7 promoter | |
| NO: 4 | | |
| Linker | GGSGG | GGCGGTTCTGGAGGT |
| SEQ ID | | |
| NO: 5 | | |
| RbsDefault | Strong RBS | TTTAAGAAGGAGATATACAT |
| SEQ ID | | |
| NO: 6 | | |
| gabDP2 | promoter | GAGATTTTGGGCTCGTCGGGGATTCGCCGGGTGCTGCAAAACC |
| SEQ ID | | ATCTACGCTCAGGACTGGGCGAGATGAAAAACTCGCTG |
| NO: 7 | | |
| Fcs | Coding | MNNEARSGSTDPGQRPRYRQVAIGHPQVQVSHVDDVLRMQPVE |
| SEQ ID | sequence | PLAPLPARLLERLVHWAQVRPDTTFIAARQADGAWRSISYVQMLA |
| NO: 8 | | DVRTIAANLLGLGLSAERPLALLSGNDIEHLQIALGAMYAGIAYCP |
| | | VSPAYALLSQDFAKLRHVCEVLTPGVVFVSDSQPFQRAFEAVLDD |
| | | SVGVISVRGQVAGRPHISFDSLLQPGDLAAADAAFAATGPDTIAKF |
| | | LFTSGSTKLPKAVITTQRMLCANQQMLLQTFPTFAEEPPVLVDWL |
| | | PWNHTFGGSHNLGIVLYNGGSFYLDAGKPTPQGFAETLRNLREI |
| | | SPTAYLTVPKGWEELVKALEQDPALREVFFARIKLFFFAAAGLSQ |
| | | SVWDRLDRIAEQHCGERIRMMAGLGMTEASPSCTFTTGPLSMAG |
| | | YVGLPAPGCEVKLVPVGDKLEARFRGPHIMPGYWRSPQQTAEAF |
| | | DEEGFYCSGDALKLADARQPELGLMFDGRIAEDFKLSSGVFVSVG |
| | | PLRNRAVLEGSPYVQDIVVTAPDRECLGLLVFPRLPECRRLAGLA |
| | | EDASDARVLANDTVRSWFADWLERLNRDAQGNASRIEWLSLLAE |
| | | PPSIDAGEITDKGSINQRAVLQRRAAQVEALYRGEDPDALHAKVRP |
| | | ...627 |
| Ech | Coding | MSKYEGRWTTVKVELEAGIAWVTLNRPEKRNAMSPTLNREMVDV |
| SEQ ID | sequence | LETLEQDADAGVLVLTGAGESWTAGMDLKEYFREVDAGPEILQE |
| NO: 9 | | KIRREASQWQWKLLRLYAKPTIAMVNGWCFGGGFSPLVACDLA |
| | | ICANEATFGLSEINWGIPPGNLVSKAMADTVGHRQSLYYIMTGKT |
| | | FDGRKAAEMGLVNDSVPLAELRETTRELALNLLEKNPVVLRAAKN |
| | | GFKRCRELTWEQNEDYLYAKLDQSRLLDTTGGREQGMKQFLDDK |
| | | SIKPGLQAYKR...276 |
| PTlpa | promoter | TTTAATTTGTTTGTTAGTTAGTTTATTTGTTGGTTTGTTTGTGTTA |
| SEQ ID | | TAATAT |
| NO: 10 | | |
| SgrS | SRNA | GATGAAGCAAGGGGGTGCCCCATGCGTCAGTTTTATCAGCACT |
| SEQ ID | | ATTTTACCGCGACAGCGAAGTTGTGCTGGTTGCGTTGGTTAAG |
| NO: 11 | | CGTCCCACAACGATTAACCATGCTTGAAGGACTGATGCAGTGG |
| | | GATGACCGCAATTCTGAAAGTTGACTTGCCTGCATCATGTGTGA |
| | | CTGAGTATTGGTGTAAAATCACCCGCCAGCAGATTATACCTGCT |
| | | GGTTTTTTTT |

The plasmids P_{GFP reporter} and P_{GFP reporter(2)} were generated by inserting PCR amplified sequence (pT7-rbs34-GFP) into backbone pBbE6k and pNO4 respectively. The plasmid P_{gabDP2-NT7Tlpa-GFP} was generated by inserting PCR amplified sequences (promoter gabDP2-rbsDefault-NT7-linker-Tlpa coil) into P_{GFP reporter} with multiple PCR steps. Similarly, Plasmids P_{J23101-NT7Tlpa-GFP} was generated by inserting PCR amplified sequences of promoter J23101. The plasmid P_{J23101-CT7Tlpa} was generated by inserting the PCR amplified sequence (J23101-rbs34-Tlpa coil-linker-CT7) into backbone pBbA8c. The plasmid P_{J23101-rbs34-T7full} was generated by inserting PCR amplified sequence (promoter J23101-rbs34-T7RNAP) into backbone pBbA8c. The plasmids P_{J23101-NT7-GFP} and P_{J23101-CT7} were generated by PCR-mediated excision of the Tlpa coiled-coil sequences from the original plasmids P_{J23101-NT7Tlpa-GFP} and P_{J23101-CT7Tlpa} respectively. The plasmids P_{J23101-NT7-GFP} and P_{J23101-CT7} were generated by PCR-mediated excision of the Tlpa coiled-coil sequences from the original plasmids P_{J23101-NT7TIpa-GFP} and P_{J23101-CT7Tlpa} respectively. While, P_{J23101-NT7Tlpa-J23101-CT7Tlpa} was generated by inserting the PCR amplified sequence (J23101-rbs34-Tlpa coil-linker-CT7) originally from P_{J23101-CT7Tlpa} into backbone P_{J23101-NT7Tlpa-GFP}. The plasmids P_{plac-NT7Tlpa} and P_{plac-NT7Tlpa*} were generated by inserting the PCR amplified sequences (rbs34-NT7-Tlpa coil) and sequence (rbs34-NT7-Tlpa truncated coil) into backbone pBbA6c respectively. P_{pBad-CT7 mutatedTlpa} and P_{pBad-CT7mutatedTlpa*} were generated by inserting PCR amplified sequences (rbs34-Tlpa coil-linker-CT7R632S) and sequence (rbs34-Tlpa truncated coil-linker-CT7R632S) in the backbone pBbE8k respectively. Each of the plasmids P_{gabDP2-NT7Tlpa-GFP} and P_{J23101-NT7Tlpa-GFP} was co-transformed with P_{J23101-CT7Tlpa} to form Thermal-T7RNAP(v1 and v2) systems. P_{J23101-NT7Tlpa-J23101-CT7Tlpa} was co-transformed with P_{GFP reporter} to form Thermal-T7RNAP(v3). P_{J23101-rbs34-T7full} was co-transformed with P_{GFP reporter} to form the full length T7-WT control. P_{J23101-NT7Tlpa-GFP} and P_{J23101-CT7Tlpa} was co-transformed to form the Split-T7 control. P_{plac-NT7Tlpa}, P_{pBad-CT7mutatedTlpa} and P_{GFP reporter(2)} were tri-transformed to form [NT7-Tlpa+CT7(R632S)-Tlpa] system. While, P_{plac-NT7Tlpa*}, P_{pBad-CT7mutatedTlpa*} and P_{GFP reporter(2)} were tri-transformed to form the [NT7-Tlpa*+CT7(R632S)-Tlpa*] system. Next, the plasmids P_{J23100-Tlpa} and P_{pTlpa-RFP} were generated by inserting PCR amplified sequences (promoter J23100-rbs34-Tlpa36) and (promoter pTlpa-rbs34-RFP) into backbones pBbA8c and pBbE6k respectively. Correspondingly, the plasmids P_{J23100-Tlpa} and P_{pTlpa-RFP} were co-transformed to form the Tlpa system. In the growth inhibition experiments, the plasmid P_{gabDP2-NT7Tlpa-pT7-sgrs-GFPreporter} was generated by inserting PCR amplified sequence (rbs34-SgrS-15T-J23101-rbs34-GFP-rrnBT1) into backbone P_{gabDP2-NT7Tlpa-GFP} with multiple PCR steps. Its control plasmid P_{gabDP2-NT7Tlpa-GFPreporter(control)} was generated by PCR-mediated excision of the sequence (promoter pT7-rbs34-GFP-15T) from the backbone P_{gabDP2-NT7Tlpa-GFP} and the subsequent insertion of sequence (promoter J23101-rbs34-GFP-rrnBT1) into the same backbone. Both of the plasmids P_{gabDP2-NT7Tlpa-pT7-sgrs-GFPreporter} and P_{gabDP2-NT7Tlpa-GFPreporter(control)} were each co-transformed with P_{J23101-CT7Tlpa} in chemical competent E.coli K-12 MG1655 cells to form the ThermalT7RNAP-SgrS system and the ThermalT7RNAP-SgrS (control) system respectively. Next, the plasmid P_{Tlpa-sgrs-RFPreporter} was generated by inserting PCR amplified sequence (SgrS-15T-J23101-rbs34) into backbone P_{pTlpa-RFP}. Its control plasmid P_{pTlpa-sgrs-RFPreporter(control)} was generated by PCR-mediated excision of the promoter sequence pTlpa from the backbone P_{pTlpa-RFP} and the subsequent insertion of new promoter J23101 into the same backbone. Both of the plasmids P_{pTlpa-sgrs-RFPreporter} and P_{pTlpa-sgrs-RFPreporter(control)} were each co-transformed with P_{J23100-Tlpa} in MG1655 cells to form the Tlpa-SgrS system and the Tlpa-SgrS (control) system respectively.

### Growth and Characterisation

All chemicals were purchased from Sigma Aldrich (Sigma, MO, USA), unless stated otherwise. All glycerol stocks were prepared by mixing 500 μL overnight culture with 500 μL of sterilized 100% glycerol. Seed cultures from glycerol stocks were inoculated 20 hours overnight in 5 mL Invitrogen Luria Broth Base LB medium (ThermoFischer, USA) and supplemented with Kanamycin sulfate (Merck, Germany) (50 μg/mL), Chloramphenicol (25 μg/mL) and Ampicillin (100 μg/mL) whenever necessary and incubated in a mini NB-205 shaking incubator (BioTek, USA) at 40° C., 225 rpm.

To run the thermal regulation assay, 100 μL of overnight cultures were added to 5 mL fresh pre-warmed LB and grown for 90 mins at 40° C. at 225 rpm. Subsequently, the refreshed cell cultures were dispensed into the wells of the 96-Well Non-Skirted PCR plate (Thermo Scientific, USA) at 100 μL. Final concentrations of 1 mM of IPTG (FirstBase, Singapore) and 0.2% L(+) arabinose were added to some cell cultures which required chemical induction. Once 96-Well PCR plate had been loaded, the plate was sealed fully with adhesive film, and left to incubate inside the thermal cycler T100 (Bio-Rad Laboratories, Hercules, CA, USA). Each row was programmed at the same temperature using the thermal ‘gradient’ function to create the desired temperature range from 30 to 42° C. After the 18 hours incubation, the cell culture in each individual well was transferred into the 96-well microplate using a multi-channel pipette. The corresponding fluorescence intensities and optical density readings were read in the H1 Synergy (BioTek, USA) at the following settings: GFP gain: 75, GFP: excitation 485 nm, emission 528 nm; OD: 600 nm.

For continuous kinetic experiments, the refreshed cell cultures were dispensed into the 96-well microplate at 300 μL each. The plate was covered with the plate lid and time series optical density and fluorescence were obtained at an interval of 10 minutes for a total duration of 12 hours and configured at double orbital shaking speed of 282 rpm continuously. Depending on the need of the studies, the temperature profiles were configured differently (at a fixed temperature of 30° C./37° C. or transitioning between the two temperatures at intervals of 2/3 hours) in the microplate reader. Prior to the experiment, the system was pre-incubated at 40° C. to ensure tight repressibility.

### Automated Colony Screening for Thermal-T7RNAP Mutants

Error-prone PCR was performed on the coiled-coil domains of the NT7 and CT7 protein fragments on plasmids P_{gabDP2-NT7Tlpa-GFP} and P_{J23101-CT7Tlpa} of the Thermal-T7RNAP(v1) system, using the GeneMorph II random mutagenesis kit (Agilent, USA). The generated PCR products were inserted back into the respective backbones using Gibson Assembly. The resultant mutant libraries were co-transformed with its un-mutated counterpart plasmid P_{gabDP2-NT7Tlpa-GFP} or P_{J23101-CT7Tlpa} respectively in DH-10 Beta cells in LB Agar. Approximately 10 rounds of mutagenesis were conducted to generate sufficient variants. After incubating overnight at 30° C., each individual colony was isolated using the Rotor HDA colony picker (Singer instruments, UK). Using the colony picker, each colony was replicative plated onto three identical 384-format agar plates. These three identical agar plates were each grown overnight at 30° C., 37° C. and 40° C. to screen for colonies with different activation and repressed temperatures. The next day, each identical colony under different temperatures was imaged in the colony picker upon illuminating with blue epifluorescence. Data processing was conducted to normalize the captured green fluorescence intensities with the corresponding size of individual colony (measured in mm₂) and each colony was sorted and ranked by the respective fold changes in fluorescence intensities between 30° C./37° C. and 30° C./40° C. Approximately 1700 colonies were screened in the library and 130 CT7 mutant colonies were shortlisted and quantified in the thermal regulation assay as described before. The thermal induction profiles generated were each fitted with a Hill's equation and correspondingly the activity and performance are quantified with various ranking indexes such as fold change between 10% and 90% of fluorescence intensities, the steepness of transition and half-activation temperatures. The sequences of the mutants are provided in Table 3.

TABLE 3
List of mutants discovered in the study. The associated GenBank accession
numbers for the coding sequences of mutant CT7 polymerases are given: Mut 1
(MW883576) and Mut 2 (MW883577).

| Genetic Element | Description | Nucleotide sequence |
|---|---|---|
| Mutated coiled-coil | Coding | GCGGTCCGGGCTGCAGAACGCCGGGTTGCGGAAG |
| domain of CT7 protein | sequence | TCACGCGTGCTGCCGGTAAACAGACCGGTGGCCAG |
| fragment | | AATGCGTAACAATAAACAGACCGGTGGCCAGAATG |
| (Mut1) | | CGTAAATCGAGGATTACCACTACGCTACGTGTAAAA |
| SEQ ID NO: 12 | | ACGAGGACCTGATGCACTTTGACGACTTGGGATCG |
| | | ACGAGAGCAGCGCGACAGACCGGTGGCCAGAATG |
| | | CGTAACAATAAACAGACCGGTGGCCAGAATGCGTA |
| | | AATCGAGGATTACCACTACGCTACGTGACTACGCTG |
| | | ATGTACAGATCCATACTGCATGTTCATTGACGATCTA |
| | | CAACGCATCACCCAGCTGGCGACAGAACGCATCACC |
| | | CAGCTGGCGACAGAACCAGTCACTGATGGCGGCGC |
| | | TTTCAGGCAATAAACAGACCGGTGGCCAGAATGCG |
| Mutated coiled-coil | Coding | GCGGTCCGGGCTGCAGAACGCCGGGTTGCGGAA |
| domain of CT7 protein | sequence | GTCACGCGTGCTGCCGGTGAACAGACCGCACAG |
| fragment | | GCAGAGCGGGAGCTGGCCGACGCCGCGCAGACA |
| (Mut2) | | GTCGACGACCTGGAAGAAAAACTGGTTGAACTGC |
| SEQ ID NO: 13 | | AGGACAGATATGACAGTTTGACGCTGGCGCTGGAG |
| | | TCAGAACGTTCACTGCGTGGTGAGGCTGAAGCCCT |
| | | GAAGCGTCAGAACCAGTCACTGATGGCGGCGCTTT |
| | | CAGGCAATAAACAGACCGGTGGCCAGAATGCG |

### Growth Inhibition Assay

Seed cultures of the GFP cells (ThermalT7RNAP-SgrS system) and the RFP cells (Tlpa-SgrS system), along with their control cells, were cultured 20 hours overnight in 5 ml of LB medium with the necessary antibiotics at their repressive temperature of 40° C. and 30° C. respectively. 50 μL of overnight cultures were added to 5 mL fresh pre-warmed M9 medium and grown for 90 mins at their repressive temperatures at 225 rpm. The M9 medium was prepared by adding 5×M9 salts, with final concentrations of 0.2% casamino acids, 100 μM CaCl₂, 2 mM MgSO₄ and 0.2% glucose into distilled water. The refreshed cell cultures were individually corrected to reach growth density of OD=0.1, before being dispensed in the 96-well microplate at their respective cell proportions into each well to form various co-cultures. The microplates were conducted in a continuous kinetic fashion at the required temperatures following the same microplate settings as described before.

### Computational Modelling

A mechanistic model represented in the form of ordinary differential equations (ODEs) was formulated to describe the kinetics of the thermal-repressible split-T7RNAP fusion protein and used to examine the different gene circuit configurations (data not shown). The same model was also employed to gain insight into the dynamic behaviors of the 3-hour and 2-hour interval OFF-ON thermal duty cycles. To better quantify transcriptional kinetics, a simple model was adopted from Motta-Mena et al. [Motta-Mena et al., Nature Chemical Biology 10:196-202 (2014)] to derive the activation (τ_{ON}) and deactivation (τ_{OFF}) time constants from the protein synthesis rate profiles.

Further, growth models were developed based upon monoculture data to predict the combined growth profiles and individual growth profiles of the cell populations within the co-cultures, seeded at different initial cell proportions and temperatures (data not shown). As an extension, a phenomenological model was developed (data not shown) to correlate the cell growth with the reporting fluorescence.

To facilitate the model development process, several ‘modules’ (consisting of promoter-rbs-GFP) were characterised using microplate reader and their time-series profiles were fed into the BioModel Selection System (BMSS) [Yeoh et al., ACS Synthetic Biology 8:1484-1497 (2019)] to identify an appropriate representative ODE model to be used for full model construction. These model parameters derived from the BMSS were estimated using a two-step optimization: differential evolution global optimizer followed by a constrained Nelder-Mead local optimizer (githubdotcom/EngBioNUS/BMSSlib). The same optimization technique was employed for parameter inference in most of the developed models including the models used in predicting the co-culture dynamics.

To better capture the uncertainties of model parameter estimates, Bayesian parameter inference method was also deployed to infer the probability distributions of some parameters estimates as opposed to the singleton estimated parameters values (data not shown). This inference approach is closely relevance to the Bayesian interpretation of probability, in which it depends on prior knowledge or beliefs, evidence, and likelihood to infer the posterior distributions of the parameter's estimates. The Metropolis-Hastings algorithm of the Markov chain Monte Carlo (MCMC) method was implemented in the parameterization process [Yildirim, Bayesian inference: Metropolis-hastings samplings (2012)], which enables one to sample from distribution without having to compute all the high dimensional integrals that demands huge computational efforts. A normal distribution is assumed for all the priors with half of the individual mean values were assigned to the individual standard deviations.

### Statistics

All data were shown as mean±S.D (n=3). All samples were prepared in technical triplicates; Statistical significance was determined by performing a two-sample unpaired t-test using Microsoft Excel (Microsoft, USA), a prior F-test was conducted to reveal equal variance or unequal variance of the samples in comparison. Blanking was included in each experiment whereby the auto-fluorescence reading of the medium was recorded. The GFP/OD₆₀₀ₙₘ reading was calculated as fluorescence of (GFPₛₐₘₚₗₑ−GFP_{blank})/OD₆₀₀ₙₘ at each time point.

### Example 2

### High Performance Thermal-Repressible T7 RNA Polymerase (Thermal-T7RNAP)

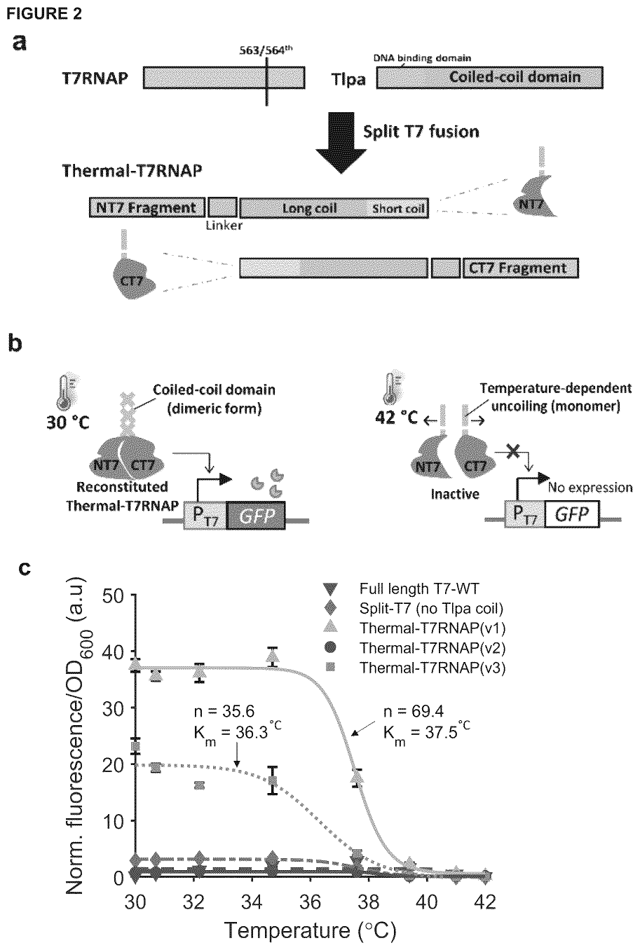

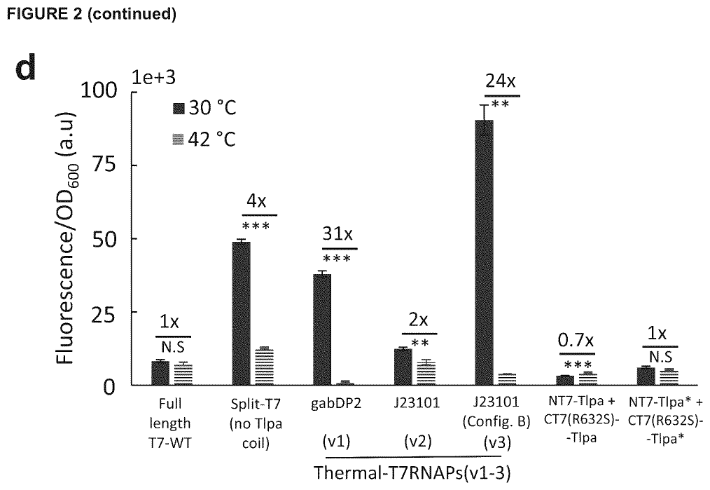

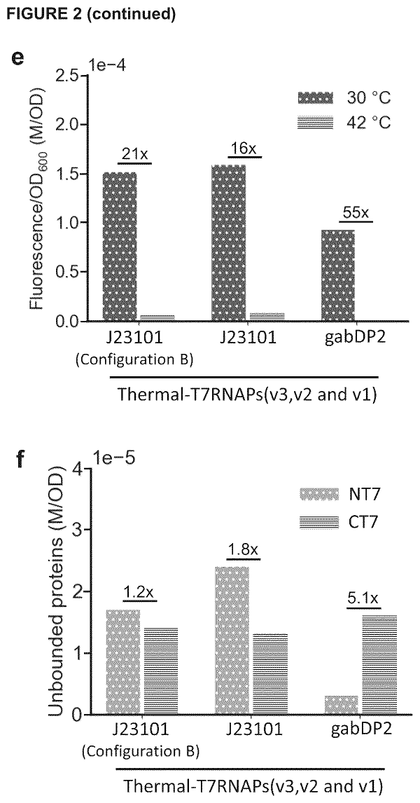

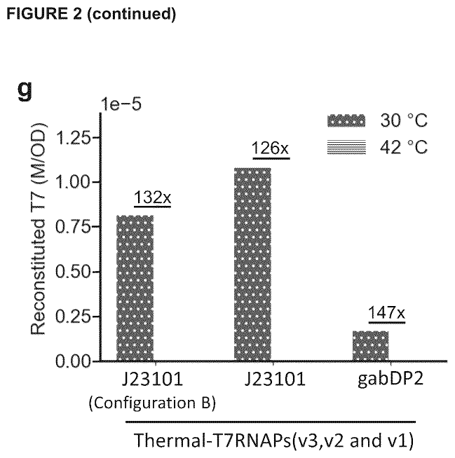

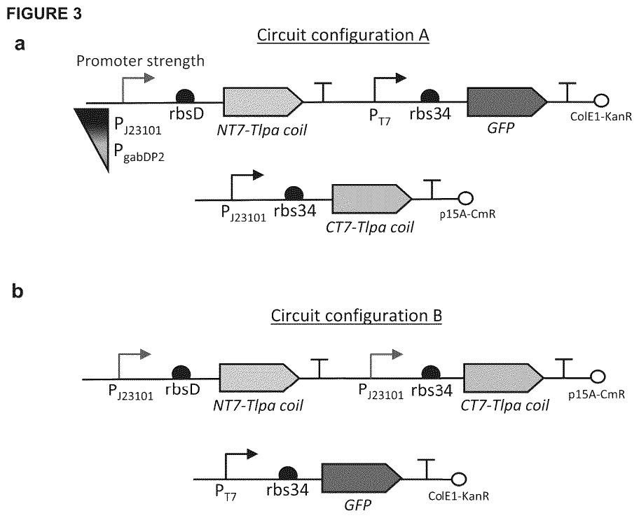

It was hypothesized that direct thermal control over the T7RNAP can be established (FIG. 2a) by leveraging the temperature-sensitive C-terminal coiled-coil domain of the Tlpa protein as its fusion partners, which exhibited temperature dependent uncoiling at high temperatures (>37° C.) [Piraner et al., Nat Chem Biol 13:75-80 (2017)]. This thermal characteristic would be valuable in bringing apart the N-terminal and C-terminal fragments of split fusion proteins at high temperatures (>40° C.), and after coiled-coil dimerization, be brought together at lower temperature of 30° C. (FIG. 2b). Recent studies have investigated ideal split sites of T7RNAP that allow modification with extra regulatory protein domains and one promising site is the 563/564 position which retained the activity of the polymerase [Baumschlager et al., ACS Synth Biol 6:2157-2167 (2017); Han et al., Biotechnology and Bioengineering 115:156-164 (2017)]. Guided by which, the inventors adopted the same split site as the position to fuse with the Tlpa's coiled-coil. To test the hypothesis, constructs in which the N-terminal based (NT7-Tlpa coil) and C-terminal based (CT7-Tlpa coil) fusion proteins were driven by constitutive promoters (gabDP2 and J23101 respectively) was first designed and built to form the Thermal-T7RNAP(v1) system (FIG. 3a).

The performance of the Thermal-T7RNAP(v1) system was studied using the thermal regulation assay. Within a biological relevant temperature range (30-42° C.) that is suitable for cell growth and for many biocatalytic processes, the thermal system exhibited a dynamic range of 31-fold between the permissive (30° C.) and restrictive (42° C.) states and displayed sharp thermal transition centred at 37.5° C. within a narrow functional range of 5° C. (FIG. 2c). The temperature response profile was well represented by a Hill equation (FIG. 2c and Table 4) and revealed the system's tightness with a maximum repression capacity, Kᵢₙₕ, of 96% and a half-activation temperature, Kₘ, of 37.5° C. which is defined as the temperature at which the fluorescence intensity is reduced to 50% of its maximum.

TABLE 4
Temperature response profile

| Name of system | n | Kₘ | Kᵢₙₕ |
|---|---|---|---|
| Full length T7-WT | 1.8990e+02 | 4.0846e+01 | 5.6382e−01 |
| Split-T7 (no Tlpa | 5.4240e+01 | 3.7661e+01 | 7.5484e−01 |
| coil) | | | |
| Thermal- | 6.9398e+01 | 3.7527e+01 | 9.5735e−01 |
| T7RNAP(v1) | | | |
| Thermal- | 1.2297e+02 | 3.8368e+01 | 4.2718e−01 |
| T7RNAP(v2) | | | |
| Thermal- | 3.5636e+01 | 3.6340e+01 | 9.7089e−01 |
| T7RNAP(v3) | | | |

^{a}The Hill equation is given as
$( 1 - {K}_{i n h} ( \frac{{T}^{n}}{{T}^{n} + {K}_{m}^{n}} ) ) ,$ where Kᵢₙₕ indicates the maximum repression capacity; Kₘ and n represent the half-activation temperature and the hill coefficient respectively.

In comparison, the fold-difference between 30° C. and 42° C. for the Split-T7 system (FIG. 2d), which expresses NT7 and CT7 protein fragments solely in the absence of the coiled-coil domains, remains low (4-fold). Importantly, its maximum repression capacity of 75% was also lower than the Thermal-T7RNAP(v1) system (96%) (Table 4), with the basal expression of the Split-T7 system being much higher than that of the Thermal-T7RNAP(v1) at 42° C. (FIG. 2d). This suggests that the Tlpa's coiled-coil domain in Thermal-T7RNAP(v1) is sufficiently strong enough to sequester the CT7 and NT7 protein fragments away from each other and provide high amount of thermal repressibility at high temperatures. In addition, when NT7-Tlpa coil was expressed in the absence of its counterpart CT7-Tlpa coil, it resulted in very minimal expression of the reporter (data not shown). This shows that the high activity of the split polymerase system requires the presence of both protein fragments.

The inventors also investigated the suitability of various lengths of the Tlpa's coiled-coil acting as fusion partners for the NT7 and CT7 protein fragments. The coiled-coil consists mainly of heptad repeats and separated into two distinctive coiled-coil regions: the longer N-terminal based 164-residues coil and the shorter C-terminal based 114-residues coil (FIG. 2a) [Mason and Ardnt, Chembiochem 5:170-176 (2004)]. By selective removal of the shorter coiled-coil (258-371^{th} residues) from the T7 fusion partners, the truncated (Tlpa*) system which retained the longer coil (94-257^{th} residues) suffered from minimal expression and no observable fold change to temperature changes (FIG. 2d). A possible explanation for its poor performance could be due to the perturbation of interfacial ionic interactions when essential amino acids residues located at the shorter coiled-coil which governed the C₂ symmetry of the parallel coiled structure are absent [Piraner, D. I., Wu, Y. and Shapiro, M. G. ACS Synthetic Biology, 8: 2256-2262 (2019)]. This suggests that engineering a high-performance temperature-sensitive split-polymerase system cannot be easily accomplished through mere excisions of conserved regions of the coiled-coil but rather allude to the need for developing a comprehensive mutagenesis workflow, in which the inventors performed, to identify consensus sequences in Tlpa that governs its thermal performance within the fusion polymerase.

Next, when compared to Thermal-T7RNAP(v1), the full length T7-WT control exhibited 5 times lower maximal expression and no visible fold-difference (FIG. 2d). The corresponding growth rate of the full length T7-WT was also 30% lower across the temperature range (data not shown). This may indicate metabolic burden and stress caused by the transcriptionally overactive wild-type polymerase [Temme et al., Nucleic Acids Res 40:8773-8781 (2012)]. The use of the mutant T7RNAP(R632S), a variant that was previously adopted in the light inducible split-T7 systems [Baumschlager et al., ACS Synth Biol 6:2157-2167 (2017)] was also investigated. However, there was minimal expression (FIG. 2d). The wild-type T7RNAP-WT polymerase was thus chosen as the fusion partners in the Thermal-T7RNAP systems.

In the process, the expression levels and the ratios were varied between the individual NT7-Tlpa coil and CT7-Tlpa coil proteins (FIG. 2d) to study how these changes affect the system's performance. Hence, Thermal-T7RNAP(v2) was constructed by replacing the constitutive promoter that controls the expression level of the NT7-Tlpa coil in Thermal-T7RNAP(v1) from gabDP2 (low) to J23101 (high), while keeping the CT7-Tlpa coil protein expression constant using J23101 on another plasmid of similar copy number (FIG. 3a). To test a different gene configuration, Thermal-T7RNAP(v3) was created by expressing NT7 and CT7 protein fragments on the same plasmid with the strong promoter J23101 (FIG. 3b). To gain insights into the different system behaviours, a mechanistic model was developed that accounted for the strength of the promoters, the plasmid copy number, and importantly, the impact of actual amino acid lengths (NT7-Tlpa coil and CT7-Tlpa coil) on mRNA and protein synthesis rates. The model was initially used to capture the behaviours of the Thermal-T7RNAP(v3) at 30° C. and 37° C. To enable the model to predict the performances of different gene circuit configurations at 30° C. (permissive state) and 42° C. (restrictive state), the inventors further incorporated the system's dose/temperature response (FIG. 2c). As a result, the model was able to predict the fluorescent protein expressions, the amount of reconstituted T7RNAP that contributes to fluorescence expression and the abundance of unbounded NT7 and CT7 protein fragments which represent inactive polymerases at the different temperatures (FIGS. 2e-g, respectively).

In general, the model predicted the fold-change of Thermal-T7RNAP(v3) fluorescence expression at 30° C. and 42° C. The relative expression levels of Thermal-T7RNAP(v3) and Thermal-T7RNAP(v1) (FIG. 2e) simulated by the model coincided with experimental fluorescence levels (FIG. 2d). Using the weak promoter gabDP2 to drive the NT7-Tlpa coil has resulted in the greatest fold-change (31-fold) among the systems which also corresponds with the model prediction showing highest fold-change for the respective promoter (FIG. 2d and e). By expressing NT7 and CT7 protein fragments on the same plasmid, the Thermal-T7RNAP(v3) exhibited a comparable fold-difference (24-fold) between 30° C. and 42° C. but achieved two times higher maximum expression, compared to Thermal-T7RNAP(v1) and also captured by the model. The system's tightness of Thermal-T7RNAP(v3) was also comparable to Thermal-T7RNAP(v1) with a maximum repression capacity of 97% and half-activation temperature of 36.3° C. (FIG. 2c and Table 4). Although the Thermal-T7RNAP(v2) was predicted to produce fluorescence expression at levels similar to the Thermal-T7RNAP(v3) system (FIG. 2e), experimentally (FIG. 2d), the system suffered from low expression and fold change, and exhibited considerable leakiness as indicated by the much lower maximum repression capacity of 43%.

The high expression levels of Thermal-T7RNAP(v3) at 30° C. could be due to better balance between the amount of NT7 and CT7 protein fragments as observed in the model (FIG. 2f). In comparison, the model revealed a five times lower abundance of NT7-Tlpa coil proteins within the Thermal-T7RNAP(v1) as driven by the weaker gabDP2 promoter (FIG. 2f), which limits the total amount of reconstituted T7RNAP to be formed at 30° C. and likely accounted for the roughly 50% lower fluorescence expression observed in experiment and from the model (FIG. 2d and e). On the other hand, the slightly higher fold change of Thermal-T7RNAP(v1) compared to Thermal-T7RNAP(v3) could be because the amount of reconstituted T7RNAP generated is within the sensitive region of the T7 promoter (FIG. 2g), thus making the system more sensitive to temperature changes. Lastly, the observed lower fluorescence expression than predicted of Thermal-T7RNAP(v2) (FIG. 2d and e), where the NT7-Tlpa coil and CT7-Tlpa coil are expressed on different plasmids by the strong J23101 promoters, could be attributed to the excessive accumulation of the NT7-Tlpa coil and reconstituted T7RNAP by more than 30% in comparison to Thermal-T7RNAP(v3) (FIG. 2f and g), ultimately affecting its system performance. Taken together, while Thermal-T7RNAP(v1) yielded the highest fold-difference, Thermal-T7RNAP(v3) was also identified which yielded the greatest maximal expression that has potential utility in applications such as bioproduction.

### Example 3

### Thermal Control of Bioproduction; Vanillin

Typically, in bioproduction, there is a need to induce the expression of the enzymes.

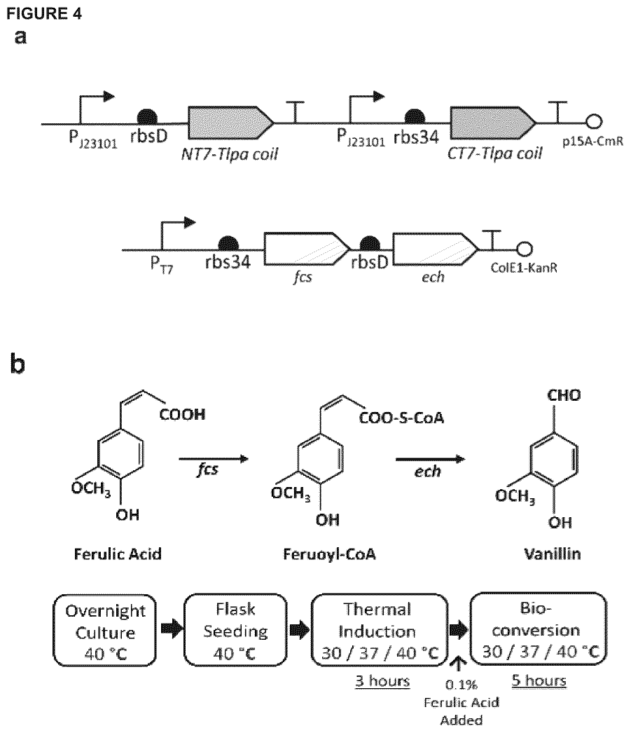

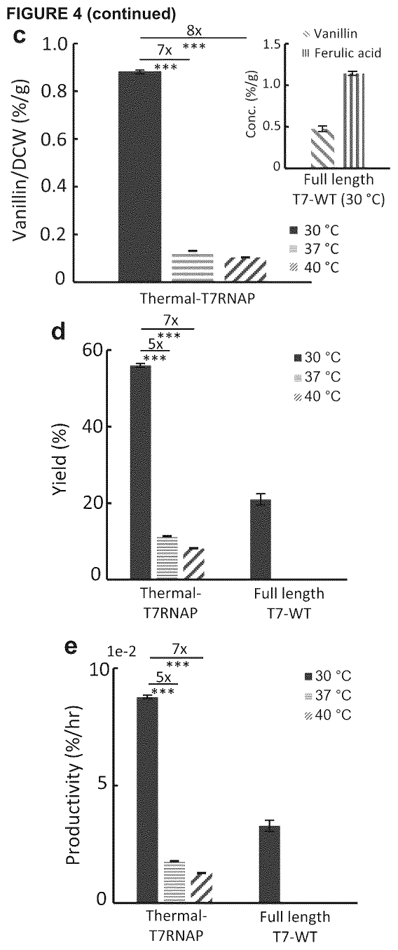

In part, the Thermal-T7RNAP system offers two key advantages: First, the decrease in temperature is already a conventional practice to enhance the product stability and yield [Qing et al., Nat Biotechnol 22:877-882 (2004)], and secondly, it offers a direct method of regulating enzyme expressions without the need of chemical inducers [Valdez-Cruz, N. A., et al., Microb Cell Fact, 9, 1-16 (2010)]. The Thermal-T7RNAP was utilised to thermally control the expression of two enzymes, feruloyl-CoA synthetase (fcs) and feruloyl-CoA hydratase (ech) located in the same operon involved in the vanillin bio-conversion pathway at 30° C. (FIG. 4a). These two enzymes are responsible for the biotransformation of ferulic acid, a common feedstock in industrial processes, into natural vanillin (FIG. 4b), a valuable form sought in food and fragrance industry as opposed to the chemically-synthesized form [Gallage, N. J. and Moller, B. L. Mol Plant, 8: 40-57 (2015)]. In a way, the Thermal-T7RNAP is suitable for the vanillin bio-conversion as its yield peaked at 30° C. [Converti, A., et al., Braz J Microbiol, 41, 519-530 (2010)], and the reduction in cultivation temperature from 37° C. to 30° C. is already part of the conventional practice to enhance the overall vanillin yield; by preventing harmful toxic proteins from overly accumulating in cells at lower temperatures [Barghini, P., et al., Microbial Cell Factories, 6: 13 (2007)], the Thermal-T7RNAP system was able to thermally control the expression of enzymes involved in the bio-conversion of ferulic acid into ‘natural’ vanillin. While a high level of vanillin was produced at the activating temperature of 30° C., minimal amounts of vanillin and associated yields were reported at the repressive temperatures of 37° C. and 40° C. (FIG. 4c, d and e).

### Example 4

### Dynamic Control of Thermal-T7RNAPs and Their Activation/Deactivation Kinetics

A key feature of an ideal thermal-switchable system is the ease of tuning the gene expression levels by means of adjustment to the temperature protocols. The dynamic study has two main objectives—firstly, to study whether temperature-controlled (re)activations of existing and de novo polymerases can generate reversible gene expression following different cooling and heating regimes and secondly, to leverage on the developed mechanistic model to offer quantitative insights into the inherent kinetics of the Thermal-T7RNAP.

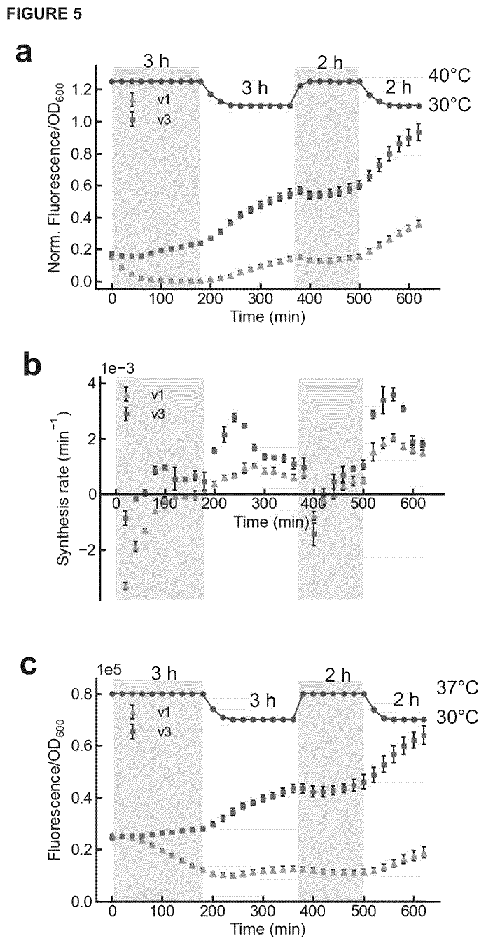

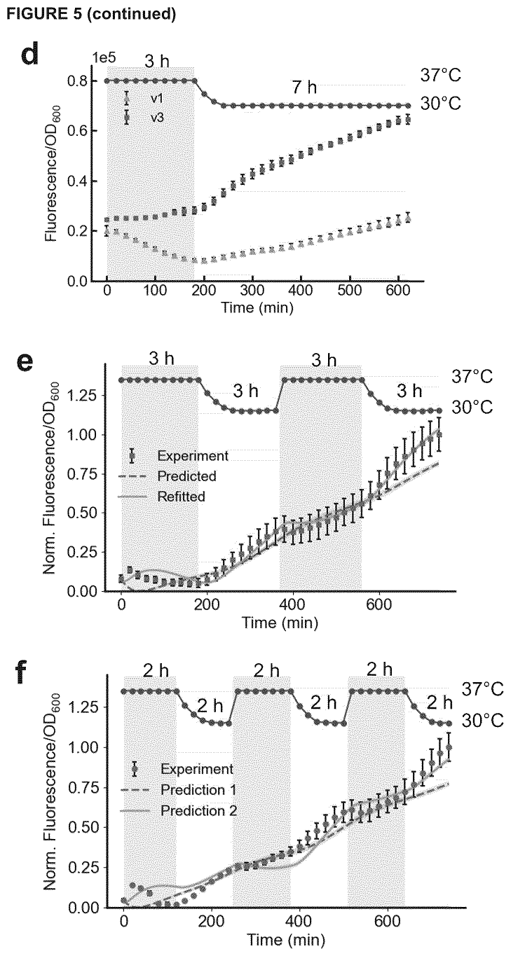

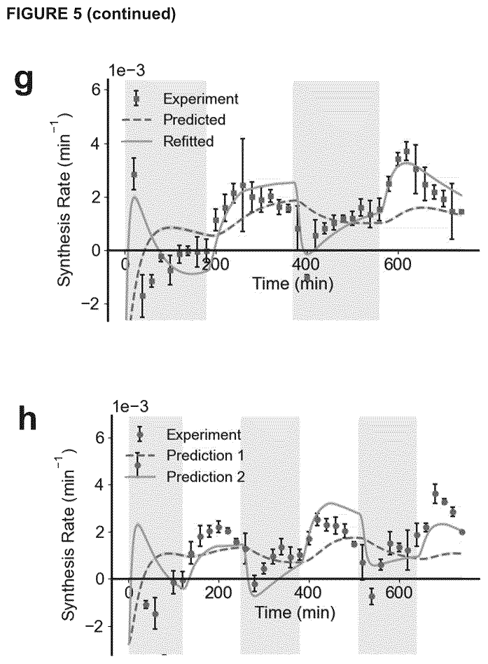

The effects of administering a patterned 3-hour (h)-3 h-2 h-2 h OFF-ON (40-30° C.) thermal duty cycle was investigated, through examining the GFP expressions (FIG. 5a) and the associated GFP synthesis rates (FIG. 5b) in the Thermal-T7RNAP(v1) and Thermal-T7RNAP(v3) systems. Both systems exhibited similar trends of increase and decrease in GFP expressions at the respective ON and OFF states (FIG. 5a). During the ON state (180-360 mins), there was a sharp increase in GFP synthesis rates for the initial 60 mins (FIG. 5b), which was more apparent for the Thermal-T7RNAP(v3), before attaining the steady synthesis rates in both systems. There was an overall increase in GFP expressions which was mediated by systems' de-repression when temperature was lowered from 40° C. to 30° C. In the subsequent OFF state (360-500 mins), when the temperature increased to 40° C., there was an almost immediate repression of GFP synthesis and cessation of GFP accumulation as indicated by the plateau features within the GFP expression profiles (FIG. 5a). Noticeably, the transient thermal regulations were highly reversible in both systems and remained sustainable over the full 10 h duration. Albeit the lower basal leakiness observed in the Thermal-T7RNAP(v1) system (FIG. 5a), the final expression level of the Thermal-T7RNAP(v3) at the end of the regime was much higher than the former.

Next, the performance of Thermal-T7RNAP(v1) and Thermal-T7RNAP(v3) systems was tested when the temperatures were altered between 37° C. and 30° C. in the patterned 3 h-3 h-2 h-2 h thermal duty cycle (FIG. 5c) or within a single-step 3 h-7 h OFF-ON protocol (FIG. 5d). These temperatures are used in conventional practices in bioproduction. When compared to previous (40-30° C.) thermal cycle study, the systems were not as fully repressed which resulted in slight basal leakiness when the lower OFF state temperature of 37° C. was applied. Nevertheless, despite the difference in time intervals, both systems were still able to achieve highly apparent transient thermal regulations in the 10 h duration and even attained comparable final GFP expression values.

The activation and deactivation kinetics of the systems were determined under the temperature cycling between permissible ON temperature (30° C.) and restrictive OFF temperature (40° C.). While in vitro measurements can directly probe into the coiling/uncoiling of alpha-helix structures [Naik, R. R., et al., Biosensors and Bioelectronics 16: 1051-1057 (2001)] in assisting split-polymerase reconstitution/sequestration but precise and reliable measurements of Thermal-T7RNAP initiating transcription are nonetheless undermined by reporter mRNA instability and slow reporter protein turnover [Motta-Mena, L. B., et al., Nature Chemical Biology 10: 196-202 (2014)]. As an alternative, a mathematical model from earlier work was adopted, which has been previously used to provide information of transcriptional kinetics based on activation/deactivation measurements [Motta-Mena, L. B., et al., Nature Chemical Biology 10: 196-202 (2014)], to obtain similar transcriptional kinetics information of the Thermal-T7RNAP systems during activation (30° C.) and deactivation phases (40° C.). From the model, under the specific temperature regime, the activation (τ_{ON}) and deactivation (τ_{OFF}) time constants represent the respective time taken to attain half-activation of the maximum steady synthesis rate and attain exponential decay to 1/e of its initial rate, as derived from the synthesis rate profiles of the 3 h-3 h-2 h-2 h OFF-ON cycle (FIG. 5b). Correspondingly, the model was able to recapitulate the dynamic behaviour of the synthesis rates. The respective estimated time constants for Thermal-T7RNAP(v1) (τ_{ON}:24 min; τ_{OFF}:16 min) and Thermal-T7RNAP(v3) (τ_{ON}:12 min; τ_{OFF}:14 min) systems fall in the order of tens of minutes. While the deactivation kinetics remained comparable in both systems, it was revealed that the activation kinetics of the Thermal-T7RNAP(v3) system was twice as fast as the Thermal-T7RNAP(v1) system, suggesting the faster dynamism of the former system to be activated in response to temperature perturbations.

The dynamic performance of Thermal-T7RNAP(v3) system was the examined under a temperature range commonly used in bioproduction (30-37° C.). The intent is to study the induction time response and the performance under different thermal duty cycles (FIG. 5e-h). Within the 3-hour time interval OFF-ON cycle which spanned 12 hours (FIG. 5e and g), during the ON state (180-360 mins), an increase in GFP expression was observed as mediated by system's de-repression. In the subsequent OFF state (360-540 mins), when the temperature increased from 30° C. to 37° C., there was an immediate repression of GFP expression. In a separate independent 2-hour interval OFF-ON cycle study, similar patterns of GFP expression and protein synthesis rate profiles were also observed and remained thermally responsive despite a reduction in the duration of time intervals (FIG. 5f and h).

A mechanistic model was developed for Thermal-T7RNAP (v3) to gain quantitative insights of its thermal-switching behavior and enable prediction of the thermal performance under different thermal duty cycles. The inventors leveraged the earlier developed mechanistic model (not shown) in predicting the dynamic behaviour of the 3-hour OFF-ON cycle. Generally, when the system was switched to the ON state (FIG. 5e), our model revealed an immediate rise in reporter protein synthesis rate prior to attaining its steady rate (FIG. 5g). When the system was switched OFF, the model indicated rapid decay in protein synthesis rate and temporary cessation of the total protein level. However, the model (‘Predicted’ line) has underestimated the fast transition kinetics of the experimental dynamic profiles (FIG. 5e and g). Interestingly, after increasing the kinetic rates describing the binding-unbinding reaction of the T7RNAP proteins, the performance of the model (‘Refitted’ line) has remarkably improved. This implies that the Thermal-T7RNAP system exhibited fast and sharp binding/unbinding kinetics, with minimum delay in response to alternating temperatures (FIG. 5g). This could be due to the fast uncoiling of the temperature-sensitive domains in sequestering the T7 protein fragments at 37° C., while allowing them to be brought together at 30° C. and in effect, manifested as distinct sharp spikes/dips in individual T7 protein fragments (inactive form) or reconstituted T7RNAP (active form) between the thermal transitions. Based on the adjusted model parameters, the T7 transcription rate was also 25% higher, possibly due to better resource allocation when the system was dynamically regulated as opposed to constant induction. In addition, the binding rate, K_{b} (bounded T7RNAP) and unbinding rate, K_{ub} (unbounded T7RNAP fragments) have increased by 30-fold. Consequently, the 50% lower association constant (K_{b}/K_{ub}) implies the accumulation of unbounded proteins over its reconstituted form which is likely to account for the improvement in system's sensitivity to temperature changes. For further validation, the adjusted model (‘Prediction 2’ line) has also demonstrated robustness in predicting the dynamic behaviour of the independent 2-hour OFF-ON cycle (FIG. 5f and h).

Using the synthesis rate profiles of 3-hour and 2-hour OFF-ON cycles, the τ_{TON} and τ_{OFF} time constants were also derived when temperatures alternated between 37° C. and 30° C. A least-squared error analysis [Motta-Mena et al., Nature Chem Bio 10:196-202 (2014)] was performed to examine the best combination of τ_{ON} and τ_{OFF} time constants while cross-referencing with experimental data from both thermal cycles (data not shown). Under this set of temperature condition and time interval patterns, the analysis showed that the estimated time constants of Thermal-T7RNAP(v3) fall within a narrow range (τ_{ON}:18-23 min and τ_{OFF}:14-29 min) with a small margin of error (2−3e⁻⁶).

Taken together, the results suggest that the Thermal-T7RNAP systems exhibited responsive and reversible ON/OFF kinetics following dynamic periods of cooling and heating.

### Example 5

### Automated Screening of Thermal-T7RNAP Mutants with Different Functional Temperature Ranges

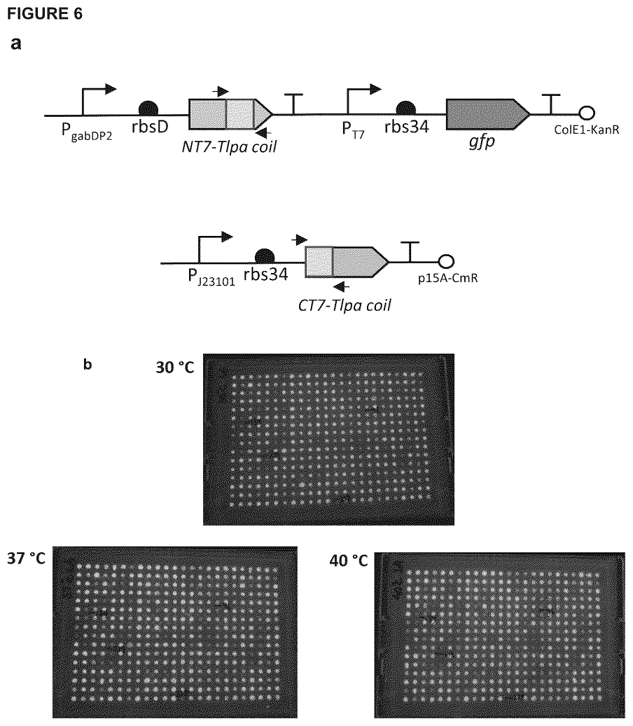

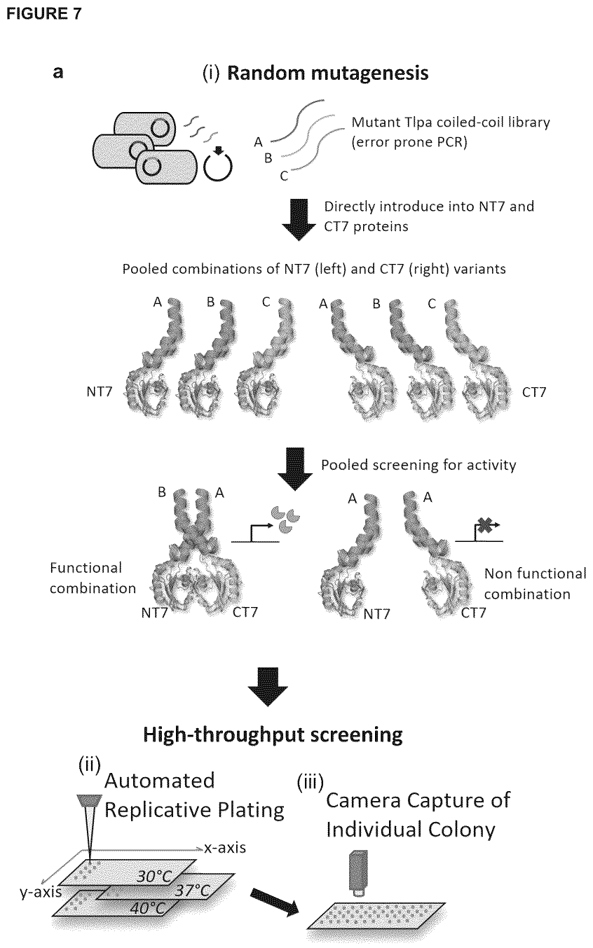

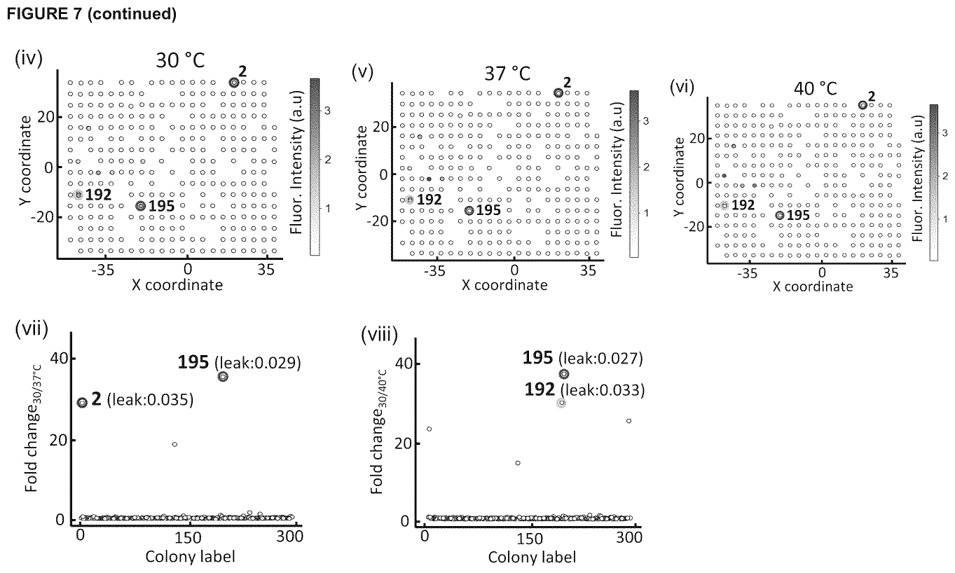

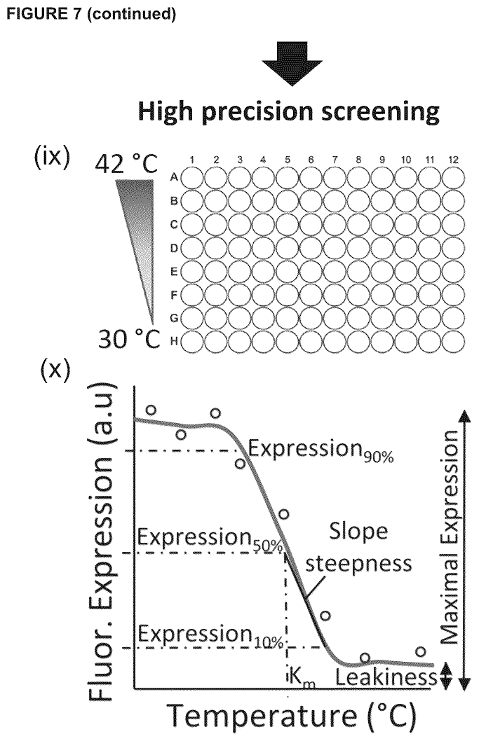

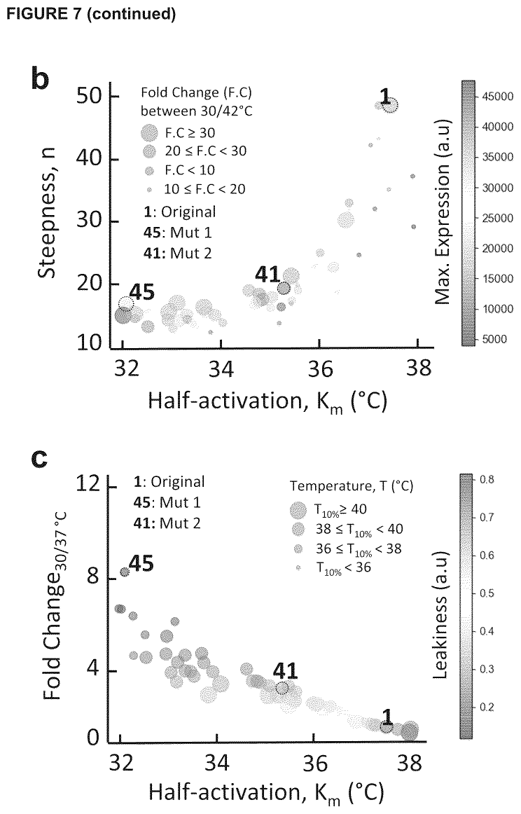

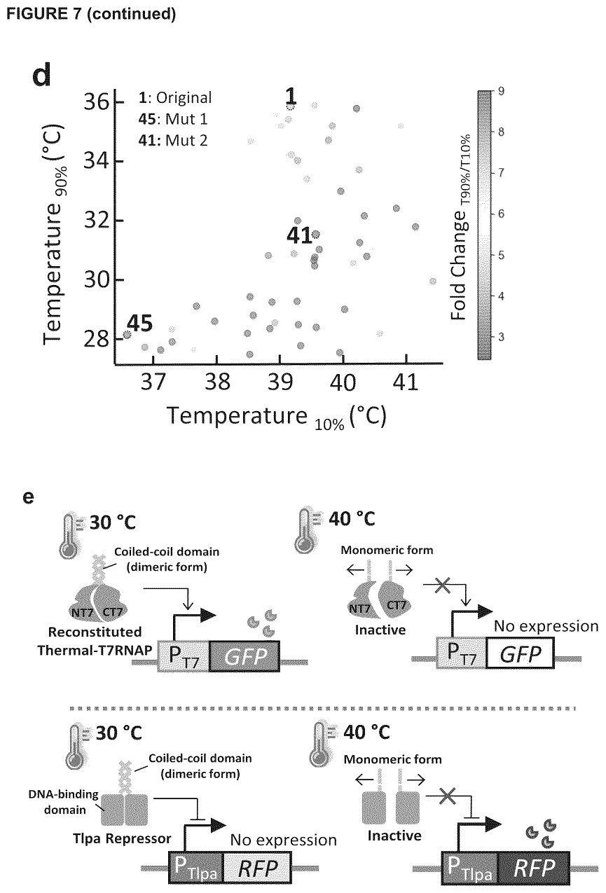

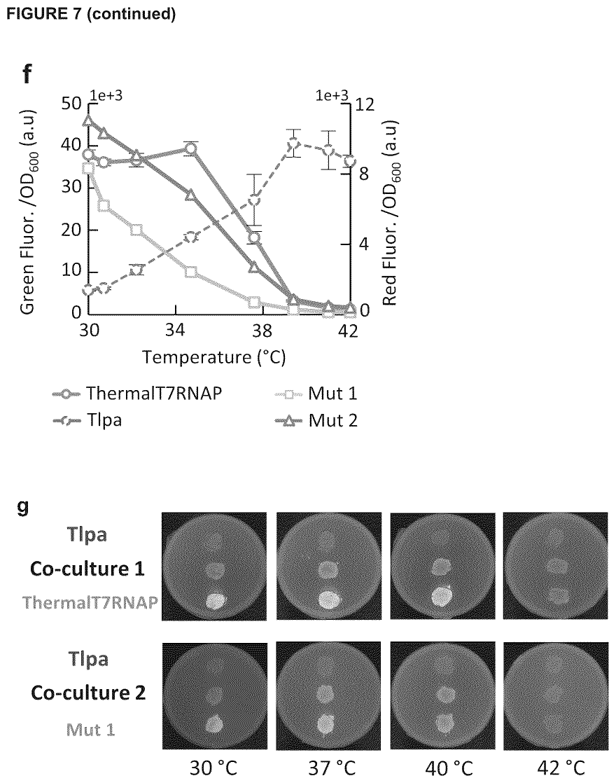

Different microbial applications require unique temperature ranges to achieve optimal activities while maintaining the system's performance. To tune the performance of the Thermal-T7RNAP and create mutants with different thermal characteristics, the coiled-coil domain of the NT7 and CT7 fusion proteins was mutated (FIG. 6) which directly influence the GFP expression at the desired repressive temperatures. In this study, the Thermal-T7RNAP(v1) was chosen as the parent backbone for ease of creating mutants (when the NT7 and CT7 fusion proteins are on separate plasmids) and more importantly this system has the highest fold change out of all the Thermal-T7RNAPs. To facilitate screening of the large library of mutants, an automated screening method was developed for the sorting and identification of ideal temperature-sensitive mutants based on the fluorescence emitted from each colony (FIG. 7a). Initially, in the high throughput screening phase, each Thermal-T7RNAP mutant was carefully ‘machine-picked’ with a colony picker and replicative-plated onto three identical 384-format agar plates which were then incubated at the various temperatures (30° C., 37° C. and 40° C.). Captured information of their overnight fluorescence outputs at respective temperatures was utilised by our in-house data-processing algorithm to sort and identify potential high-performance mutants with desirable fold differences between the permissive (30° C.) and restrictive temperatures (37° C. and 40° C.). Potential candidates were furthered characterised using liquid-cultures over the full range of temperatures (30-42° C.). In the process, their individual thermal profiles were automatically fitted with Hill equations and ranked according to their thermal performance parameters such as the half-activation temperature, Kₘ, the sharpness of the thermal transition as represented by T_{10%}-T_{90%}, the temperature difference (T) between 10% and 90% of maximal fluorescence expression, the associated fold change (Fold) and leakiness (Leak) at various temperatures.

Using the high throughput system, over 1700 colonies were screened and many exhibited constitutive expressions regardless of temperature change while others exhibited loss of expression (FIG. 6b). It was discovered that mutating the NT7 fusion protein only or simultaneously mutating both the CT7 and NT7 fusion pair yielded very few transformants (˜200) and did not exhibit desired thermal characteristics. Nonetheless, 130 CT7 mutants were isolated which exhibited thermal repressibility in the desired temperature range and were subjected to the liquid-culture characterisations of their temperature profiles in the second screening phase (FIG. 7a). Two mutants (Mut 1 and Mut 2) were identified that retained the ideal characteristic of the original Thermal-T7RNAP(v1) system but with distinctive shift in their temperature functional ranges (K_{m, original}=37.5° C., K_{m, Mut1}=32.2° C. and K_{m, Mut2}=35.4° C.) (FIG. 7b). The Mut 2 system also exhibited 20% higher maximal expression when compared to the original system. While both mutants had more gentle thermal transitions ([T_{10%}-T_{90%}]_{original}=3.3° C., [T_{10%}-T_{90%}]_{Mut1}=8.5° C. and [T_{10%}-T_{90%}]_{Mut2}=8.0° C.) (FIG. 7d), lower leakiness were exhibited by the mutants at 37° C. ([Leak_{30° C.-37° C.}]_{original} =0.68, [Leak_{30° C.-37° C.}]_{Mut1}=0.12 and [Leak_{+° C.-37° C.}]_{Mut2}=0.31) (FIG. 7c). Correspondingly, greater fold differences between 30° C. and 37° C. in Mut 1 and Mut 2 were observed ([Fold_{30° C.-37° C.}]_{original} 150, [Fold_{30° C.-37° C.}]_{Mut}=8.53 and [Fold_{30° C.-37° C.}]_{Mut}2=3.27) (FIG. 7c). Further analysis on the mutated coiled-coil domains led to the identification of five consensus regions that are potentially vital for the robust thermal behaviours of the fusion proteins.

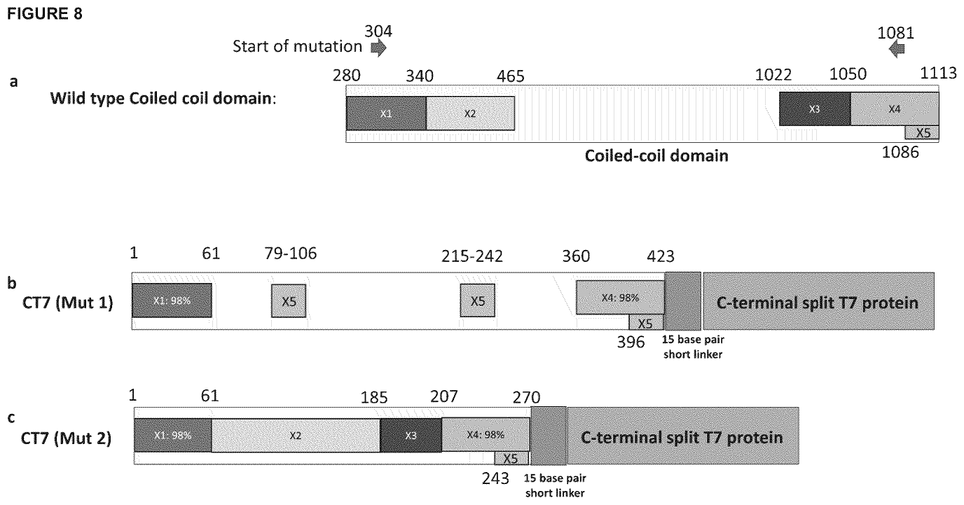

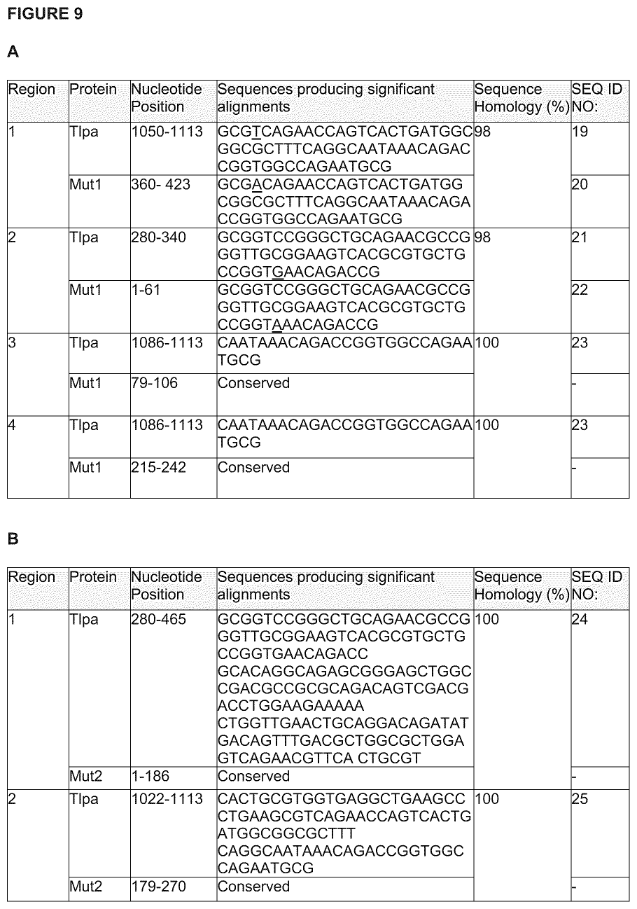

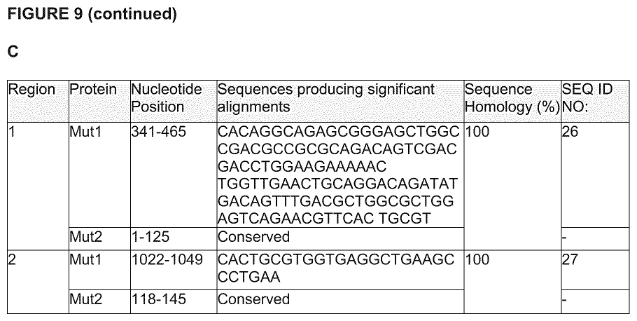

In the analysis of the mutation sites within the CT7 protein fragments, due to the effects of random mutations, we observed the selected mutants (Mut 1 and Mut 2) contained varying truncated lengths of the coiled-coil domains which were attached to their respective split polymerase units (FIG. 8). This can potentially account for their enhanced thermal performances (in terms of improved maximal expression and reduced leakiness) as they harboured shorter variants of the CT7 polymerase units; and possibly reduced the cellular burden by expressing shorter but still functional fusion proteins. After detailed alignments of the sequences (FIG. 9A-C), five consensus nucleotide regions were identified within the full length of the Tlpa's coiled coil that remained preserved within the mutant proteins (>98% homology) (FIG. 8 and Table 5).

TABLE 5
consensus nucleotide regions within the mutant proteins

| | Nucleotide positions | | |
|---|---|---|---|
| | (reference to original | | SEQ ID |
| Domain | Tlpa protein) | Nucleotide Sequence | NO. |
| X1 | 280-340 | GCGGTCCGGGCTGCAGAACGCCGGGTT | 14 |
| | | GCGGAAGTCACGCGTGCTGCCGGTGAA | |
| | | CAGACCG | |
| X2 | 341-465 | CACAGGCAGAGCGGGAGCTGGCCGAC | 15 |
| | | GCCGCGCAGACAGTCGACGACCTGGA | |
| | | AGAAAAACTGGTTGAACTGCAGGACAG | |
| | | ATATGACAGTTTGACGCTGGCGCTGGA | |
| | | GTCAGAACGTTCACTGCGT | |
| X3 | 1022-1049 | CACTGCGTGGTGAGGCTGAAGCCCTGA | 16 |
| X4 | 1050-1113 | GCGTCAGAACCAGTCACTGATGGCGGC | 17 |
| | | GCTTTCAGGCAATAAACAGACCGGTGG | |
| | | CCAGAATGCG | |
| X5 | 1086-1113 | CAATAAACAGACCGGTGGCCAGAATGCG | 18 |

Specifically, it was discovered that the X1 and X4 domains which are located at either ends of the coiled-coil were present in both the wild-type and the mutated fusion proteins. It is likely that these preserved regions are highly essential to allow proper folding and guidance of the split polymerase units. The further left shifting of the temperature transition (measured in Kₘ) for Mut 1 can be likely accounted for by the short but prominent X5 domains that were highly repeated and interspersed throughout the coiled-coil region (FIG. 8). It is important to note that while mutagenesis was performed previously on the full length of original Tlpa repressor (which contained the DNA binding and coiled-coil domains) which led to the identification of two mutant variants of Tlpa (Tlpa₃₆ and Tlpa₃₉) with performances centred at 36° C. and 39° C. respectively [Piraner, D. I., et al., Nat Chem Biol, 13: 75-80 (2017)]. However, their discovered points of mutations within the coiled-coil domain did not overlap with our study.

The ability of simultaneously thermally activating and inhibiting expressions in different cell populations can be a powerful tool in biotechnology. As a proof of concept, the heat-repressible Thermal-T7RNAP system was coupled to control GFP expression in one cell and the heat-inducible Tlpa system to control RFP expression in another cell (FIG. 7e). The thermal logic is illustrated by both thermal systems which functioned in counter-unison to produce higher GFP expressions at low temperatures (<34° C.) and higher RFP expressions (due to de-repression of Tlpa repressor from promoter pTlpa) at high temperatures (>38° C.) (FIG. 7f). The two Thermal-T7RNAP mutant systems (with smaller Kₘ) in the thermal profiles have left-shifted their temperature intersections with the Tlpa system. Similarly, in the ‘Traffic light’ agar patterns (FIG. 7g), shifting of the intersection was portrayed by the diminishing intensity of the ‘orange zone’ in the Mut 1/Tlpa co-culture when compared to the original co-culture at higher temperatures (>40° C.).

### Example 6

### Directing Microbial Community Distribution with Thermal Control

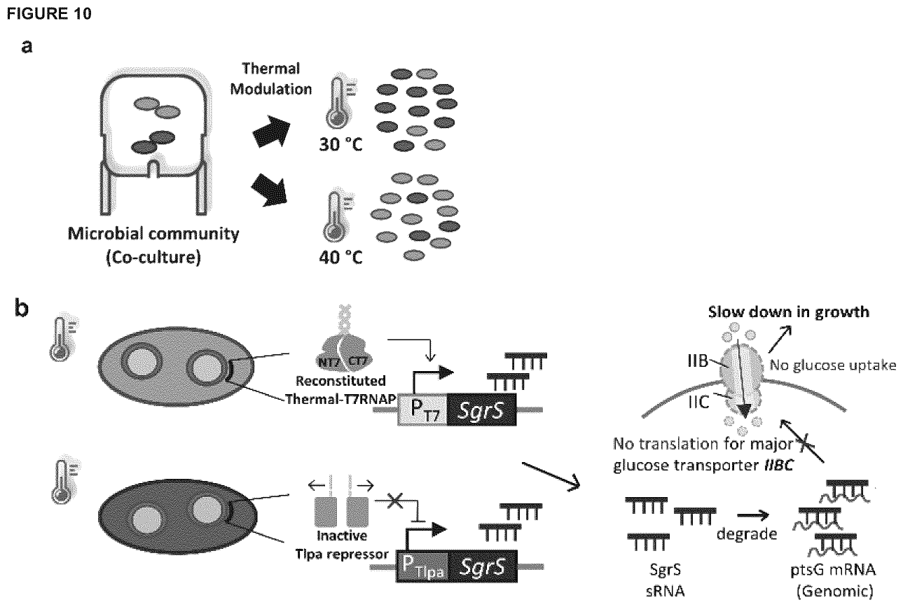

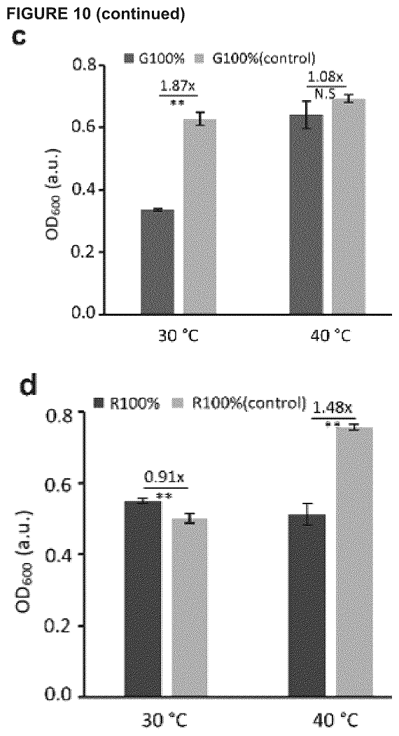

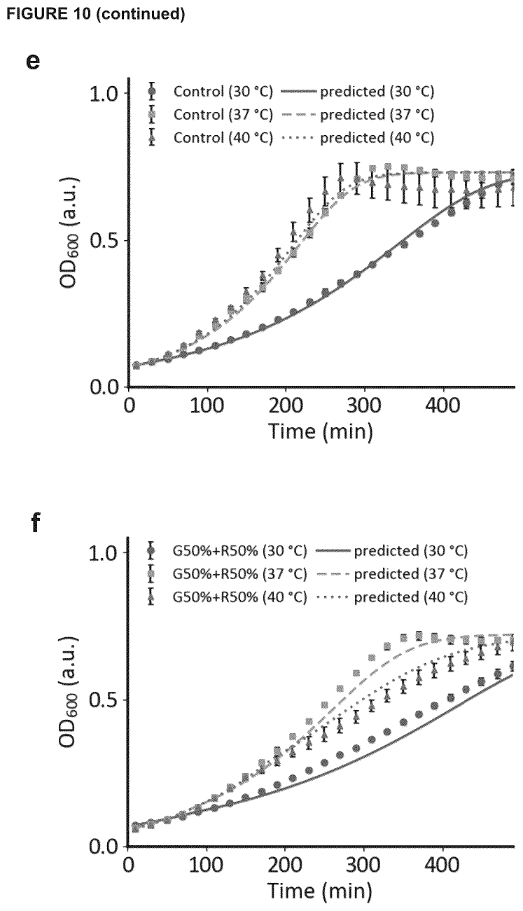

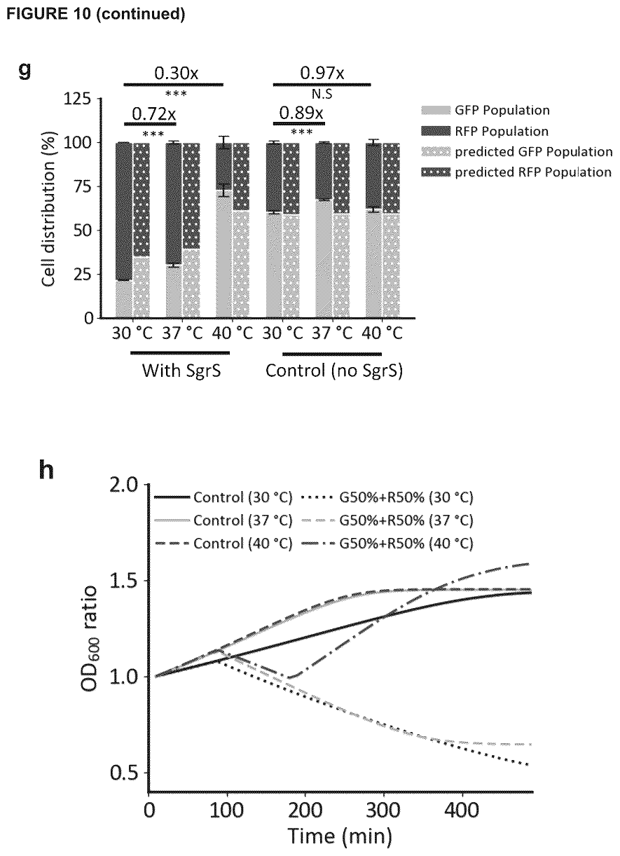

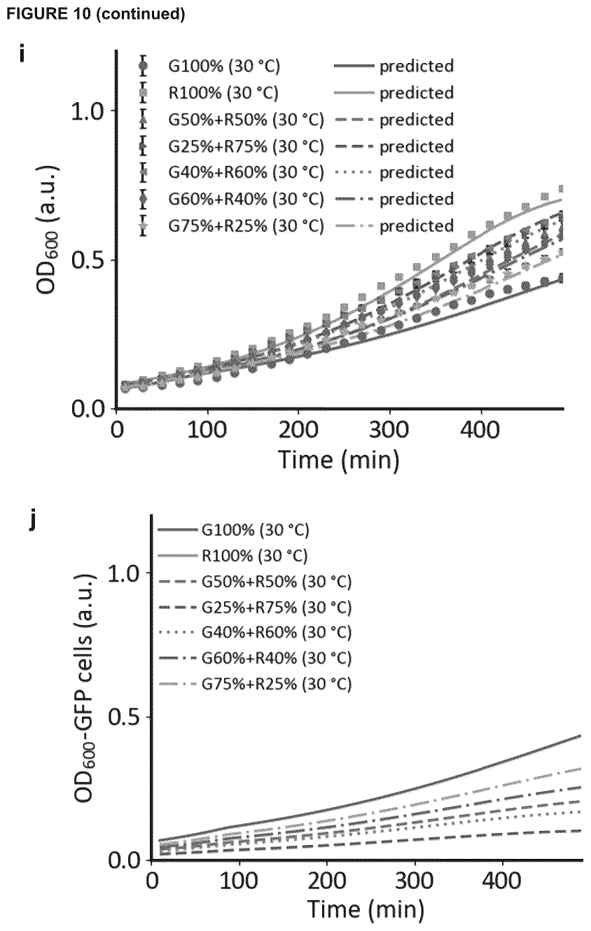

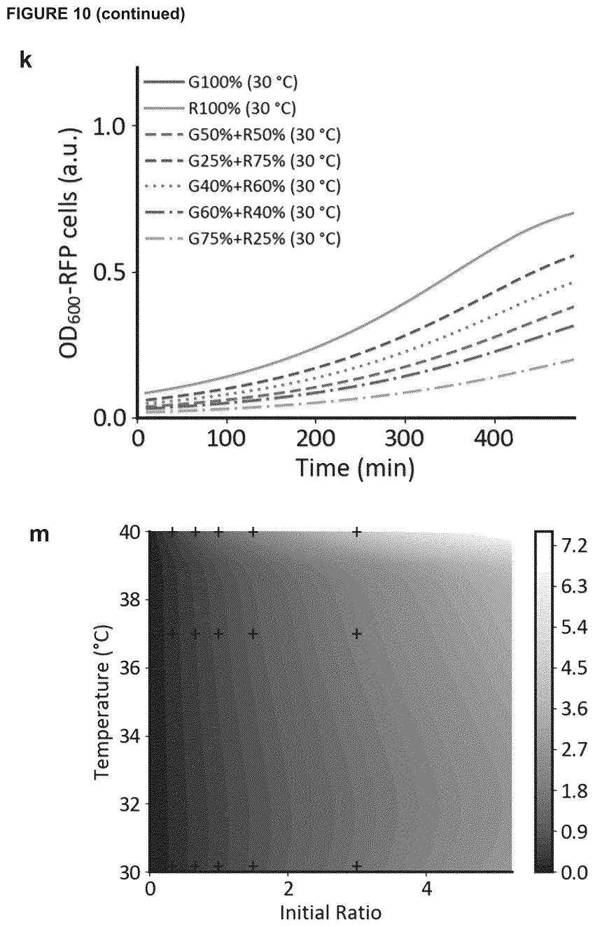

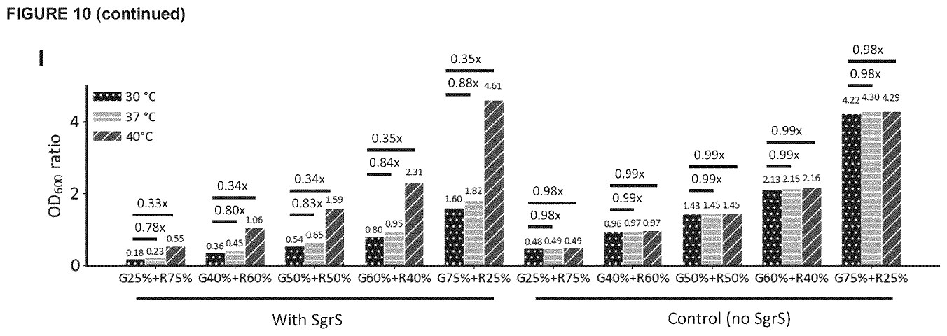

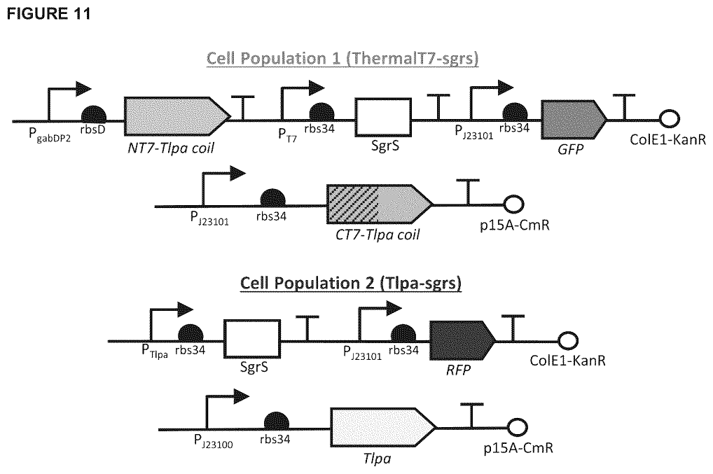

The use of thermal biosensors in controlling cell distribution within microbial community is still not well undertaken. While existing study leveraged optimal temperature ranges to enable native microbial species to co-exist [(Krieger et al., Biotechnology and Bioengineering 118:1-12 (2021)], there was a lack of capability in active control of the individual population. To address the need, the inventors developed temperature-based genetic circuits which forms thermal logic to enable thermal modulation of the growth of two engineered E. coli populations within a co-culture (FIG. 10a), in which one population is producing GFP while another is producing RFP. A thermal-repressible ThermalT7RNAP-SgrS system was built for the GFP-reporting cell (GFP_{ThermalT7RNAP-SgrS}) and a thermal-inducible Tlpa-SgrS system for the RFP-reporting cell (RFP_{Tlpa-SgrS}) (FIG. 10b and FIG. 11). Both thermal circuits were designed to slow down cellular growth by limiting glucose uptake at their ‘ON’ thermal states, by expressing the SgrS sRNA (sugar transport related silencing RNA) which functions to degrade ptsG mRNA that encodes major glucose transporter, IICB^{Glc} [(Negrete et al., Microb Cell Fact 9:75 (2010)].

Independent characterisation of the ThermalT7RNAP-SgrS and the Tlpa-SgrS systems was first conducted to study the growth inhibition profiles at different temperatures (FIG. 10c and d). At 30° C., there was an 87% decrease in the growth of the GFP_{ThermalT7RNAP-sgrs} cells based on the final OD values when compared to its corresponding control that had no SgrS (FIG. 10c). But at 40° C., there was no observable growth inhibition, showing the tightness of the system in preventing SgrS expression. Conversely, at 40° C., the RFP_{Tlpa-SgrS} cells displayed a 48% reduction in growth compared to its control but exhibited normal growth at 30° C., implying strong repression of SgrS expression at 30° C. (FIG. 10d). In parallel, a growth model was developed to investigate the effects of temperature variations on cell growth and changes in glucose uptake due to the expression of SgrS in the monocultures. In the model, the maximum growth capacity of the monoculture was presumed to be constrained by the amount of glucose carbon source. The RFP control cells were slower in growth in comparison to GFP control cells with maximum specific growth rates of 0.014 min⁻¹ and 0.016 min⁻¹ respectively (data not shown). The growth rates of both control cells also increased with increasing temperatures and defined by half-activation temperature of 29.24° C. and steepness of 10.92. The glucose sensitivity was estimated by the model to be 0.759 g/L with a yield coefficient of 0.122. By introducing SgrS expression at either 30° C. or 40° C. for the GFP_{ThermalT7RNAP-SgrS} and RFP_{Tlpa-Sgrs} cells respectively, the effects of their growth inhibition were clearly visible in both cells by an approximately 7 to 8-fold reductions in glucose sensitivity compared to their controls which led to reduced glucose uptake (data not shown). An initial delay (first 200 mins) was observed in glucose uptake inhibition for the RFP_{Tlpa-SgrS} cells but was absent from the GFP_{ThermalT7RNAP-SgrS} cells, which again demonstrated the fast thermal response of the Thermal-T7RNAP in driving the SgrS expression.

Next, both GFP_{ThermalT7RNAP-SgrS} and RFP_{Tlpa-sgrS} cells were co-cultured at similar initial ODs and mixed at equal proportion (G50%+R50%) to demonstrate that the proportion of the cells can be actively controlled using the thermal genetic circuits (i.e., the final cell distributions can be varied). The growth model developed from the monoculture was used to predict the individual growth profiles of the two cell populations within the co-culture. The growth of the two cell populations was presumed to be constrained by the shared carbon source. Their combined growth was thus computed as the sum of the individual cell populations. As evidenced, the predicted combined growth profiles of the co-culture (G50%+R50%) at different temperatures agreed well with the experimental results (FIG. 10e and f). This implies that the growth model could serve as a means to predict the actual growth profiles of the individual cell population within a co-culture.

While fluorescence-reporting is commonly used to estimate the individual cell growths within a co-culture [Nikolic N. et al., BMC Microbiology 13:258 (2013)], it was observed that the correlations between the reporting fluorescence and OD values were different even for cells with similar genetic makeup. This indicates that the use of fluorescence reporting level to estimate the individual cell growths within a co-culture may not be a good representative metric. Hence, for better representation, the inventors derived the final cell distributions of the co-cultures under different temperatures from both the experimentally measured fluorescence and the model-predicted individual growth profiles (FIG. 10g). At 30° C., the cell distributions for both experiment and prediction have exhibited prominent decline in the GFP_{ThermalT7RNAP-SgrS} population from the initial G50%+R50% to attain final ratios of [G22%+R78%]_{Expt.} and [G35%+R65%]_{Predict.} respectively (FIG. 10g). At 37° C., the final ratios of [G30%+R70%]_{Expt.} and [G39%+R61%]_{Predict.} attained were comparable to 30° C., indicating that the growth of the GFP_{ThermalT7RNAP-SgrS} population was still inhibited. This suggests that there was still significant amount of SgrS expression within the GFP_{ThermalT7RNAP-SgrS} population. It was at 40° C. when GFP_{ThermalT7RNAP-SgrS} cells had outgrown the RFP_{Tlpa-SgrS} cells; to attain final ratios of [G73%+R27%]_{Expt.} and [G61%+39%]_{Predict.} This reiterated that the ThermalT7RNAP-SgrS system remained tightly repressed at high temperatures which allowed the GFP_{ThermalT7RNAP-SgrS} cells to grow normally while the Tlpa-SgrS system produced the desired slowdown in the growth of RFP_{Tlpa-Sgrs} cells. RFP For the control co-culture, the final distributions hovered around [G60%+R40%]_{Expt. & Predict.,} across all the temperatures tested which implied a roughly 10% increase in GFP control cells even though both GFP and RFP control cells were initially cultured at equal proportion.

To reveal changes in cell proportions over time, the OD ratios were computed through dividing the predicted ODs of the GFP cells with the RFP cells (FIG. 10h). In the control co-culture (FIG. 10h), there was an increase in the OD ratios from 1.0 (equal initial cell proportions) to 1.4 eventually; showing the faster growth rate of GFP control cells. In the co-cultures with the thermal gene circuits (FIG. 10h), at 30° C. and 37° C., there were reductions in the OD ratios over time to finally attain ratios of 0.5 and 0.6 respectively. At 40° C., there was a slight decrease in the first 200 mins followed by the steady increase in the OD ratio before reaching 1.6. The initial dip in OD ratio was largely due to the mentioned delay in SgrS expression of RFP_{Tlpa-SgrS} cells while its 14% increase in final ratios compared to control co-culture was attributed to the decline in growth of RFP_{Tlpa-SgrS} cells as a result of SgrS expression at high temperatures.

Next, the effect of various initial cell proportions in the final cell distributions within the co-cultures was determined (FIG. 10i-k). The fitted growth model of the monocultures successfully predicted their combined growth profiles under different temperatures (FIG. 10i). At different initial proportions for control co-cultures, there is a constant 40% increase from their initial ratios (data not shown) and their final cell ratios remained similar at different temperatures (FIG. 10l). In the co-cultures with the thermal genetic circuits, by lowering or raising the temperature, the cell ratios were modulated as much as −50% and 65% respectively. From the model, we also identified the fine-tuning conditions required to obtain specific cell proportions (FIG. 10m), and discovered that the final cell proportions can effectively be maintained (within a 10% deviation) with respect to their initial proportions at 39.5° C.

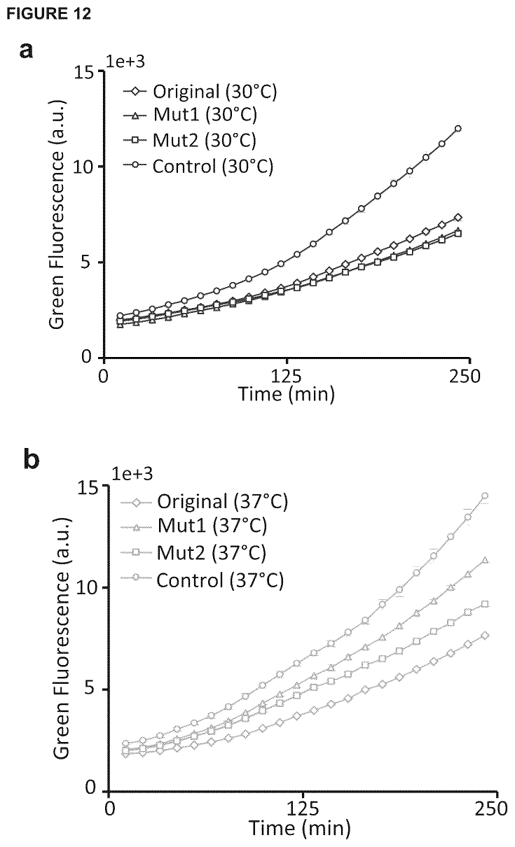

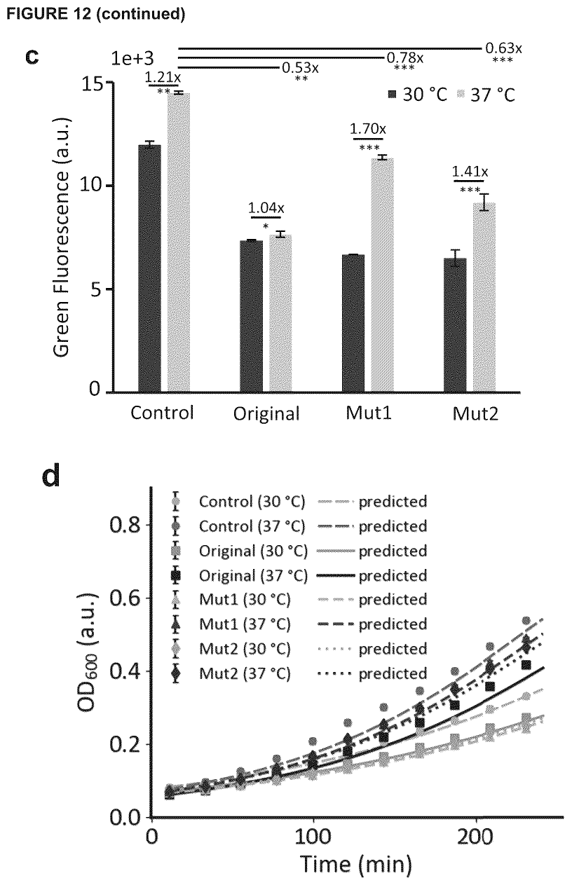

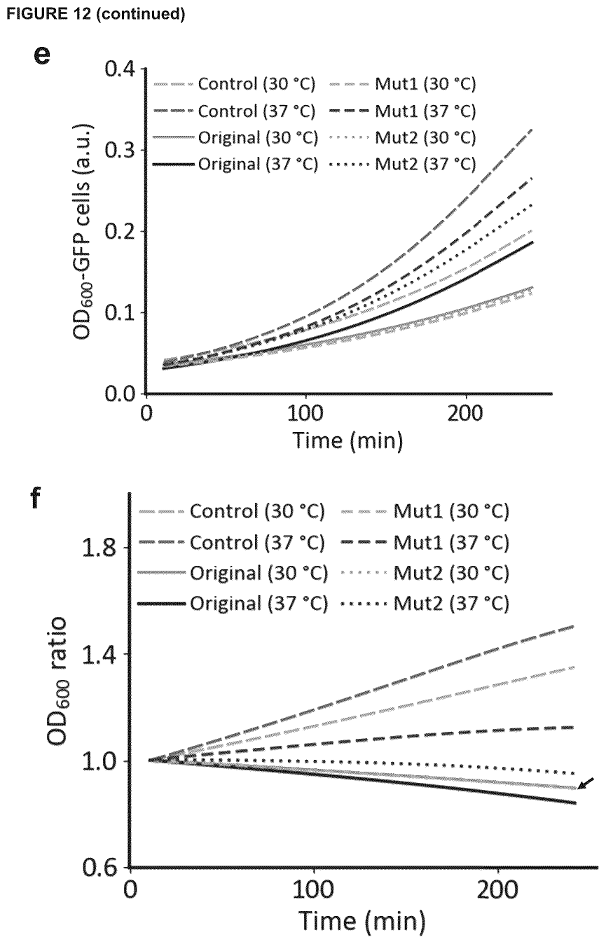

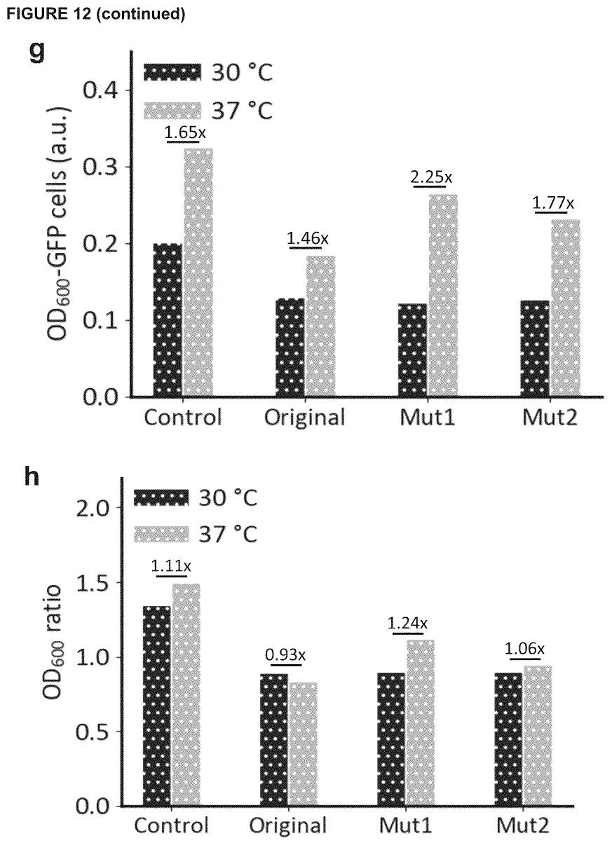

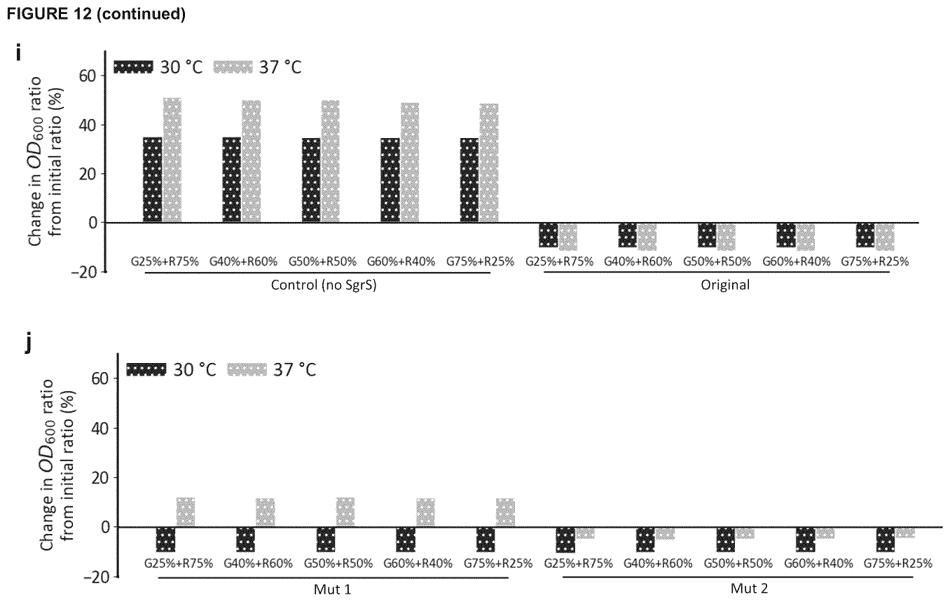

To expand the tunability of thermal modulation in co-culture, the inventors leveraged the discovered Thermal-T7RNAP CT7 mutants (Mut 1 and Mut 2) to regulate the SgrS expression in the GFP cells (FIG. 12a-c). In the earlier results, both mutants have distinctive left shifting of activation temperature (FIG. 7f) that can potentially provide better repression capability at 37° C. which is a common temperature for cell cultivation.

In the original G50%+R50% co-culture, the GFP_{ThermalT7RNAP-SgrS} cells were cultured together with the RFP_{Tlpa-SgrS} cells at equal proportion. Results show that at 30° C., the GFP_{ThermalT7RNAP-SgrS} cells exhibited the desired growth inhibition as portrayed by the 63% decrease in fluorescence intensity from the GFP control cells (FIG. 12a and c). However, at 37° C., there was still a significant 47% reduction in the fluorescence intensity from the control (FIG. 12c) which indicated that growth inhibition was still present at this higher temperature. For Mut 1 and Mut 2 co-cultures which contained the GFP_{ThermalT7RNAP-SgrS(Mut1)} and GFP_{ThermalT7RNAP-SgrS(Mut2)} respectively, at 37° C., the fluorescence reductions from the GFP control cell were lower (22% and 37% respectively) (FIG. 12c), which suggests that the mutant systems exhibited greater relief from SgrS expression at 37° C. while still achieving desirable growth inhibition at 30° C.

Similarly, we predicted the growth profiles of the mutant co-cultures at different initial cell proportions (FIG. 12d and e). The relief in SgrS inhibition was also visible from the predicted final OD values of the GFP cells (FIG. 12g) where GFP_{ThermalT7RNAP-SgrS(Mut1)} cells achieved 125% increase in cell growth at 37° C. from 30° C., followed by 77% and 46% increase for GFP_{ThermalT7RNAP-SgrS(Mut2)} and the GFP_{ThermalT7RNAP-SgrS} respectively. While the original system exhibited decrease in the OD ratio at 37° C., the ratios for Mut 1 and Mut 2 systems were maintained over time (FIG. 12f and h) and the same is true for the other initial cell proportions (FIG. 12i and j).

### SUMMARY

In the thermogenetic toolkit, existing heat-repressible systems are limited in parts [(Hoynes-O'Connor A. et al., Nucleic Acids Res 43:6166-6179 (2015); Liang R. et al., J Microbiol Methods 68:497-506 (2007); Qing G. et al., Nat Biotechnol 22:877-882 (2004); Yang Z. et al., Nucleic Acids Res 47:e137 (2019)] and restricted to thermal sensing mechanisms which rely mainly on RNA or transcription factors; and face potential issues such as functioning at low temperatures (<30° C.) which are non-optimal for cell growth and bio-catalytic processes, have wide temperature transitions, suffer delays or complex in design. To address the limitations and provide an alternative mode of thermal regulation, the present invention provides novel heat-repressible Thermal-T7 RNA polymerase (Thermal-T7RNAP) systems by fusing Tlpa coiled-coil domain with split-T7RNAP to introduce direct temperature control into the polymerase. The Examples show that the polymerase activity is well repressed at high temperatures (>40° C.) and highly suggests the NT7 and CT7 protein fragments can be sequestered by the temperature-sensitive coiled-coil domain at high temperatures (FIG. 2). The system has several advantages over existing heat-repressible systems. Firstly, the system is compact and highly simplified in design which encourages ease of incorporation into larger and more complex genetic networks to perform integrative functions. Secondly, the system is responsive and highly reversible, which is useful for transient thermal regulation of gene expressions. Thirdly, the system's temperature transition is narrow (5-8° C.) and functions within a tunable temperature range (30-42° C.) which is optimal for cell growth and bio-catalytic processes. Lastly, as the Thermal-T7RNAP system utilised the T7 phage polymerase within its design core, it can decouple transcription from the host's machinery and presume to be highly suited in performing independent functions within a large group of organisms. As an exemplification of novel thermal application, it is herein demonstrated that E. coli cell population distributions in a co-culture can be actively altered using temperature control (FIGS. 10 and 12).

One unique feature of the Thermal-T7RNAP system is the ability to undergo sharp two-way thermal switching, which is likely attributed to the highly sensitive temperature-dependent coiling and uncoiling of the coiled-coil domain (FIG. 5). The model unveiled a beneficial 50% reduction in association constant when the system was regulated dynamically. This implied a larger pool of unbounded NT7 and CT7 protein fragments would be readily available for reconstitution in the upcoming activation phase and led to the estimated half-activation response within tens of minutes. This suggests that the thermally responsive characteristic of Thermal-T7RNAP system is potentially useful in temperature-based dynamic regulation of bio-processes for example in bioproduction relevant range (30-37° C.) such as in the control of metabolic fluxes (Harder B. J. et al., Biotechnology and Bioengineering 115:156-164 (2018)], that can complement with existing light-based systems (Zhao E. M. et al., Nat Chem Biol 15:589-597 (2019)].

### REFERENCES

Abedi, M. H., Lee, J., Piraner, D. I., and Shapiro, M. G. (2020). Thermal Control of Engineered T-cells. ACS Synthetic Biology 9, 1941-1950. 10.1021/acssynbio.0c00238.
Aucoin, M. G., McMurray-Beaulieu, V., Poulin, F., Boivin, E. B., Chen, J., Ardelean, F. M., Cloutier, M., Choi, Y. J., Miguez, C. B., and Jolicoeur, M. (2006). Identifying conditions for inducible protein production in E. coli: combining a fed-batch and multiple induction approach. Microb Cell Fact 5, 27. 10.1186/1475-2859-5-27.
Barghini, P., Di Gioia, D., Fava, F. and Ruzzi, M. (2007) Vanillin production using metabolically engineered Escherichia coli under non-growing conditions. Microbial Cell Factories, 6, 13.
Baumschlager, A., Aoki, S. K., and Khammash, M. (2017). Dynamic Blue Light-Inducible T7 RNA Polymerases (Opto-T7RNAPs) for Precise Spatiotemporal Gene Expression Control. ACS Synth Biol 6, 2157-2167. 10.1021/acssynbio.7b00169.
Briand, L., Marcion, G., Kriznik, A., Heydel, J. M., Artur, Y., Garrido, C., Seigneuric, R., and Neiers, F. (2016). A self-inducible heterologous protein expression system in Escherichia coli. Sci Rep 6, 33037. 10.1038/srep33037.
Chao, Y. P., Law, W., Chen, P. T. and Hung, W. B. (2002) High production of heterologous proteins in Escherichia coli using the thermo-regulated T7 expression system. Appl Microbiol Biotechnol, 58, 446-453.
Chen, F., and Wegner, S. V. (2020). Blue-Light-Switchable Bacterial Cell-Cell Adhesions Enable the Control of Multicellular Bacterial Communities. ACS Synth Biol 9, 1169-1180. 10.1021/acssynbio.0c00054.
Converti, A., Aliakbarian, B., Dominguez, J. M., Bustos Vázquez, G. and Perego, P. (2010) Microbial production of biovanillin. Braz J Microbiol, 41, 519-530.
Dunstan, M. S., Robinson, C. J., Jervis, A. J., Yan, C., Carbonell, P., Hollywood, K. A., Currin, A., Swainston, N., Feuvre, R. L., Micklefield, J., et al. (2020). Engineering Escherichia coli towards de novo production of gatekeeper (2S)-flavanones: naringenin, pinocembrin, eriodictyol and homoeriodictyol. Synthetic Biology 5, 1-11. 10.1093/synbio/ysaa012.
Fang, Y., Wang, J., Ma, W., Yang, J., Zhang, H., Zhao, L., Chen, S., Zhang, S., Hu, X., Li, Y., and Wang, X. (2020). Rebalancing microbial carbon distribution for L-threonine maximization using a thermal switch system. Metabolic Engineering 61, 33-46. https://doi.org/10.1016/j.ymben.2020.01.009.
Fu, L., Gong, J., Gao, B., Ji, D., Han, X., and Zeng, L. (2020). Controlled expression of lysis gene E by a mutant of the promoter pL of the thermo-inducible λc1857-pL system. Journal of applied microbiology, 1-10.
Gallage, N. J. and Moller, B. L. (2015) Vanillin-bioconversion and bioengineering of the most popular plant flavor and its de novo biosynthesis in the vanilla orchid. Mol Plant, 8, 40-57.
Gal-Mor, O., Valdez, Y., and Finlay, B. B. (2006). The temperature-sensing protein TlpA is repressed by PhoP and dispensable for virulence of Salmonella enterica serovar Typhimurium in mice. Microbes Infect 8, 2154-2162. 10.1016/j.micinf.2006.04.015.
Gamboa, L., Phung, E. V., Li, H., Meyers, J. P., Hart, A. C., Miller, I. C., and Kwong, G. A. (2020). Heat-Triggered Remote Control of CRISPR-dCas9 for Tunable Transcriptional Modulation. ACS Chemical Biology 15, 533-542. 10.1021/acschembio.9b01005.
Goers, L., Freemont, P., and Polizzi, K. M. (2014). Co-culture systems and technologies: taking synthetic biology to the next level. J R Soc Interface 11, 1-13. 10.1098/rsif.2014.0065.
Greco, F. V., Grierson, C. S., and Gorochowski, T. E. (2020). Harnessing the central dogma for stringent multi-level control of gene expression. bioRxiv, 2020.2007.2004.187500. 10.1101/2020.07.04.187500.
Guarino, A., Fiore, D., Salzano, D., and di Bernardo, M. (2020). Balancing Cell Populations Endowed with a Synthetic Toggle Switch via Adaptive Pulsatile Feedback Control. ACS Synth Biol 9, 793-803. 10.1021/acssynbio.9b00464.
Grunberg, R. and Serrano, L. (2010) Strategies for protein synthetic biology. Nucleic Acids Res, 38, 2663-2675.
Han, T., Chen, Q., and Liu, H. (2017). Engineered Photoactivatable Genetic Switches Based on the Bacterium Phage T7 RNA Polymerase. ACS Synth Biol 6, 357-366. 10.1021/acssynbio.6b00248.
Harder, B. J., Bettenbrock, K., and Klamt, S. (2018). Temperature-dependent dynamic control of the TCA cycle increases volumetric productivity of itaconic acid production by Escherichia coli. Biotechnology and bioengineering 115, 156-164. 10.1002/bit.26446.
Hoynes-O'Connor, A., Hinman, K., Kirchner, L., and Moon, T. S. (2015). De novo design of heat repressible RNA thermosensors in E. coli. Nucleic Acids Res 43, 6166-6179. 10.1093/nar/gkv499.
Hussaina, F., Guptab, C., Hirninga, A. J., Ottb, W., Matthewsa, K. S., Josi, K., and Bennett, M. R. (2014). Engineered temperature compensation in a synthetic genetic clock. PNAS 111, 972-977.
Keto-Timonen, R., Hietala, N., Palonen, E., Hakakorpi, A., Lindstrom, M., and Korkeala, H. (2016). Cold Shock Proteins: A Minireview with Special Emphasis on Csp-family of Enteropathogenic Yersinia. Front Microbiol 7, 1151. 10.3389/fmicb.2016.01151.
Kortmann, J., and Narberhaus, F. (2012). Bacterial RNA thermometers: molecular zippers and switches. Nature Reviews Microbiology 10, 255-265. 10.1038/nrmicro2730.

Krieger, A. G., Zhang, J., and Lin, X. N. (2021). Temperature regulation as a tool to program synthetic microbial community composition. Biotechnology and bioengineering 118, 1-12. https://doi.org/10.1002/bit.27662.
Liang, R., Liu, X., Liu, J., Ren, Q., Liang, P., Lin, Z., and Xie, X. (2007). A T7-expression system under temperature control could create temperature-sensitive phenotype of target gene in Escherichia coli J Microbiol Methods 68, 497-506. 10.1016/j.mimet.2006.10.016.
Lin, X., Krieger, A. and Zhang, J. (2020) Temperature regulation as a tool to program synthetic microbial community composition.
Liu, C. C., Jewett, M. C., Chin, J. W., and Voigt, C. A. (2018). Toward an orthogonal central dogma. Nat Chem Biol 14, 103-106. 10.1038/nchembio.2554.
Miller, I. C., Gamboa Castro, M., Maenza, J., Weis, J. P., and Kwong, G. A. (2018). Remote Control of Mammalian Cells with Heat-Triggered Gene Switches and Photothermal Pulse Trains. ACS Synth Biol 7, 1167-1173. 10.1021/acssynbio.7b00455.
Motta-Mena, L. B., Reade, A., Mallory, M. J., Glantz, S., Weiner, O. D., Lynch, K. W., and Gardner, K. H. (2014). An optogenetic gene expression system with rapid activation and deactivation kinetics. Nature Chemical Biology 10, 196-202. 10.1038/nchembio.1430.
Naik, R. R., Kirkpatrick, S. M., and Stone, M. O. (2001). The thermostability of an α-helical coiled-coil protein and its potential use in sensor applications. Biosensors and Bioelectronics 16, 1051-1057. https://doi.org/10.1016/S0956-5663(01)00226-3.
Naseri, G., and Koffas, M. A. G. (2020). Application of combinatorial optimization strategies in synthetic biology. Nature Communications 11, 2446. 10.1038/s41467-020-16175-y.
Negrete, A., Ng, W. I., and Shiloach, J. (2010). Glucose uptake regulation in E. coli by the small RNA SgrS: comparative analysis of E. coli K-12 (JM109 and MG1655) and E. coli B (BL21). Microb Cell Fact 9, 75. 10.1186/1475-2859-9-75.
Nikolic, N., Barner, T., and Ackermann, M. (2013). Analysis of fluorescent reporters indicates heterogeneity in glucose uptake and utilization in clonal bacterial populations. BMC Microbiology 13, 258. 10.1186/1471-2180-13-258.
Nistala, G. J., Wu, K., Rao, C. V., and Bhalerao, K. D. (2010). A modular positive feedback-based gene amplifier. Journal of Biological Engineering 4, 4. 10.1186/1754-1611-4-4.
Pearce, S. C., McWhinnie, R. L., and Nano, F. E. (2017). Synthetic temperature-inducible lethal gene circuits in Escherichia coli. Microbiology 163, 462-471. 10.1099/mic.0.000446.
Peng, Y. Y., Howell, L., Stoichevska, V., Werkmeister, J. A., Dumsday, G. J., and Ramshaw, J. A. M. (2012). Towards scalable production of a collagen-like protein from Streptococcus pyogenes for biomedical applications. Microb Cell Fact 11, 1-8.
Piraner, D. I., Abedi, M. H., Moser, B. A., Lee-Gosselin, A., and Shapiro, M. G. (2017). Tunable thermal bioswitches for in vivo control of microbial therapeutics. Nat Chem Biol 13, 75-80. 10.1038/nchembio.2233.

Piraner, D. I., Wu, Y., and Shapiro, M. G. (2019). Modular Thermal Control of Protein Dimerization. ACS Synthetic Biology 8, 2256-2262. 10.1021/acssynbio.9b00275.
Qing, G., Ma, L. C., Khorchid, A., Swapna, G. V., Mal, T. K., Takayama, M. M., Xia, B., Phadtare, S., Ke, H., Acton, T., et al. (2004). Cold-shock induced high-yield protein production in Escherichia coli. Nat Biotechnol 22, 877-882. 10.1038/nbt984.
Rodrigues, J. L., Couto, M. R., Araújo, R. G., Prather, K. L. J., Kluskens, L., and Rodrigues, L. R. (2017). Hydroxycinnamic acids and curcumin production in engineered Escherichia coli using heat shock promoters. Biochemical Engineering Journal 125, 41-49. https://doi.org/10.1016/j.bej.2017.05.015.
Rodrigues, J. L., and Rodrigues, L. R. (2018). Potential Applications of the Escherichia coli Heat Shock Response in Synthetic Biology. Trends Biotechnol 36, 186-198. 10.1016/j.tibtech.2017.10.014.
Roell, G. W., Zha, J., Carr, R. R., Koffas, M. A., Fong, S. S., and Tang, Y. J. (2019). Engineering microbial consortia by division of labor. Microbial Cell Factories 18, 35. 10.1186/s12934-019-1083-3.
Salila Vijayalal Mohan, H. K., Chee, W. K., Li, Y., Nayak, S., Poh, C. L., and Thean, A. V. Y. (2020). A highly sensitive graphene oxide based label-free capacitive aptasensor for vanillin detection. Materials & Design 186, 108208. https://doi.org/10.1016/j.matdes.2019.108208.
Schramm, T., Lempp, M., Beuter, D., Sierra, S. G., Glatter, T., and Link, H. (2020). High-throughput enrichment of temperature-sensitive argininosuccinate synthetase for two-stage citrulline production in E. coli. Metabolic Engineering 60, 14-24. 10.1016/j.ymben.2020.03.004.
Segall-Shapiro, T. H., Meyer, A. J., Ellington, A. D., Sontag, E. D., and Voigt, C. A. (2014). A ‘resource allocator’ for transcription based on a highly fragmented T7 RNA polymerase. Mol Syst Biol 10, 742. 10.15252/msb.20145299.
Sen, S., Apurva, D., Satija, R., Siegal, D., and Murray, R. M. (2017). Design of a Toolbox of RNA Thermometers. ACS Synthetic Biology 6, 1461-1470. 10.1021/acssynbio.6b00301.
Shah, N. H., and Muir, T. W. (2014). Inteins: Nature's Gift to Protein Chemists. Chem Sci 5, 446-461. 10.1039/C3SC52951G.
Singh, R., Kumar, M., Mittal, A., and Mehta, P. K. (2016). Microbial enzymes: industrial progress in 21st century. 3 Biotech 6, 174-174. 10.1007/s13205-016-0485-8.
Sorensen, H. P., and Mortensen, K. K. (2005). Soluble expression of recombinant proteins in the cytoplasm of Escherichia coli. Microb Cell Fact 4, 1. 10.1186/1475-2859-4-1.
Stirling, F., Bitzan, L., O'Keefe, S., Redfield, E., Oliver, J. W. K., Way, J., and Silver, P. A. (2017). Rational Design of Evolutionarily Stable Microbial Kill Switches. Mol Cell 68, 686-697 e683.10.1016/j.molcel.2017.10.033.
Temme, K., Hill, R., Segall-Shapiro, T. H., Moser, F., and Voigt, C. A. (2012). Modular control of multiple pathways using engineered orthogonal T7 polymerases. Nucleic Acids Res 40, 8773-8781. 10.1093/nar/gks597.
Valdez-Cruz, N. A., Caspeta, L., Pérez, N. O., Ramirez, O. T., and Trujillo-Roldán, M. A. (2010). Production of recombinant proteins in E. coli by the heat inducible expression system based on the phage lambda pL and/or pR promoters. Microb Cell Fact 9, 1-16.
Wang, W., Li, Y., Wang, Y., Shi, C., Li, C., Li, Q., and Linhardt, R. J. (2018). Bacteriophage T7 transcription system: an enabling tool in synthetic biology. Biotechnol Adv 36, 2129-2137.10.1016/j.biotechadv.2018.10.001.
Wang, Z. W., Law, W. S. and Chao, Y. P. (2004) Improvement of the thermoregulated T7 expression system by using the heat-sensitive lacl. Biotechnology progress, 20, 1352-1358.
Wei, Y., Murphy, E. R., Larramendy, M., and Soloneski, S. (2016). Temperature-dependent regulation of bacterial gene expression by RNA thermometers. Nucleic Acids—from Basic Aspects to Laboratory Tools Specific. IntechOpen, London, 157-181.
Xu, S., Wang, Q., Zeng, W., Li, Y., Shi, G., and Zhou, J. (2020). Construction of a heat-inducible Escherichia coli strain for efficient de novo biosynthesis of I-tyrosine. Process Biochemistry 92, 85-92. https://doi.org/10.1016/j.procbio.2020.02.023.
Yang Zheng, Fankang Meng, Zihui Zhu, Weijia Wei, Zhi Sun, Jinchun Chen, Bo Yu, Chunbo Lou, and Chen, G.-Q. (2019). A tight cold-inducible switch by coupling thermosensitive transcriptional and proteolytic regulatory parts. Nucleic Acids Research 47, e137. 10.1093/nar/gkz785.
Yeoh, J. W., Ng, K. B. I., Teh, A. Y., Zhang, J., Chee, W. K. D., and Poh, C. L. (2019). An Automated Biomodel Selection System (BMSS) for Gene Circuit Designs. ACS Synthetic Biology 8, 1484-1497. 10.1021/acssynbio.8b00523.
Yildirim, I. (2012). Bayesian inference: Metropolis-hastings sampling (Dept. of Brain and Cognitive Sciences, Univ. of Rochester, Rochester, NY).
Zhang, C., Seow, V. Y., Chen, X., and Too, H.-P. (2018). Multidimensional heuristic process for high yield production of astaxanthin and fragrance molecules in Escherichia coli. Nature Communications 9, 1858. 10.1038/s41467-018-04211-x.
Zhao, E. M., Suek, N., Wilson, M. Z., Dine, E., Pannucci, N. L., Gitai, Z., Avalos, J. L., and Toettcher, J. E. (2019). Light-based control of metabolic flux through assembly of synthetic organelles. Nat Chem Biol 15, 589-597. 10.1038/s41589-019-0284-8.

1. An isolated heat-repressible Split-T7 polymerase fusion protein, comprising:
(i) a split T7 RNA polymerase polypeptide (T7RNAP);
(ii) a polypeptide coiled-coil domain; and
(iii) a linker peptide between the polypeptides (i) and (ii),
wherein an N-terminal fragment of T7 RNA polymerase (T7RNAP) is fused to a polypeptide coiled-coil domain and a C-terminal fragment of T7 RNA polymerase is fused to a polypeptide coiled-coil domain.

2. The isolated heat-repressible Split-T7 polymerase fusion protein of claim 1, wherein the coiled-coil domain is selected from the group comprising TlpA polypeptide, M class C proteins from group A streptococci, such as Arp4 and Sir22, and Hv1/VSOP voltage-gated H⁺ channel protein; and/or
wherein the N-terminal fragment of T7 RNA polymerase and the C-terminal fragment of T7 RNA polymerase are derived by splitting the T7 RNA polymerase polypeptide at amino acid position 563/564 of the mature peptide sequence.

3. (canceled)

4. The isolated heat-repressible Split-T7 polymerase fusion protein of claim 2, wherein the T7RNAP comprises an amino acid sequence with at least 90% sequence identity with the amino acid sequence is set forth in SEQ ID NO: 1 and/or the TlpA coiled-coil comprises an amino acid sequence with at least 90% sequence identity with the amino acid sequence set forth in SEQ ID NO: 3.

5. The isolated heat-repressible Split-T7 polymerase fusion protein of claim 1, wherein the coiled-coil domain fused to the C-terminal fragment of T7 RNA polymerase comprises one or more domains, X1, X2, X3, X4 and X5, encoded by polynucleotide sequences having at least 70%, at least 80%, at least 90% or 100% identity with sequences selected from the group comprising X1 (SEQ ID NO: 14), X2 (SEQ ID NO: 15), X3 (SEQ ID NO: 16), X4 (SEQ ID NO: 17) and X5 (SEQ ID NO: 18) or combinations thereof.

6. The isolated heat-repressible Split-T7 polymerase fusion protein of claim 5, wherein the coiled-coil domain fused to the C-terminal fragment of T7 RNA polymerase comprises a X1 domain at the N-terminal end and a X4 domain at the C-terminal end of the coiled-coil domain; and/or
wherein the polynucleotide sequence set forth in X1 (SEQ ID NO: 14) comprises a G/A substitution at position 52 and/or the polynucleotide sequence set forth in X4 (SEQ ID NO: 17) comprises a T/A substitution at position 4.

7. (canceled)

8. The isolated heat-repressible Split-T7 polymerase fusion protein of claim 6, wherein the coiled-coil domain fused to the C-terminal fragment of T7 RNA polymerase comprises a plurality of X5 domains; and/or
wherein the coiled-coil domain fused to the C-terminal fragment of T7 RNA polymerase consists of, from N-terminal to C-terminal, domains X1, X2, X3 and X4 or X5.

9. The isolated heat-repressible Split-T7 polymerase fusion protein of claim 8, wherein the coiled-coil domain fused to the C-terminal fragment of T7 RNA polymerase comprises, from N-terminal to C-terminal, domains X1, X5, X5 and X4 or X5.

10. The isolated heat-repressible Split-T7 polymerase fusion protein of claim 9, wherein the coiled-coil domain fused to the C-terminal fragment of T7 RNA polymerase is encoded by a polynucleotide sequence comprising the sequence set forth in SEQ ID NO: 12.

11. (canceled)

12. The isolated heat-repressible Split-T7 polymerase fusion protein of claim 10, wherein the coiled-coil domain fused to the C-terminal fragment of T7 RNA polymerase is encoded by a polynucleotide sequence comprising the sequence set forth in SEQ ID NO: 13.

13. The isolated heat-repressible Split-T7 polymerase fusion protein of claim 5, wherein, as a result of varying combinations of coiled-coil domains X1, X2, X3, X4 and/or X5, the active temperature range can be tuned.

14. The isolated heat-repressible Split-T7 polymerase fusion protein of claim 1, wherein the Split-T7 polymerase is active at temperatures in the range of about 30° C. to about 39° C. and is thermally repressed above 39° C.

15. An isolated nucleic acid molecule capable of expressing the fusion protein of claim 1.

16. A plasmid or vector comprising the nucleic acid molecule of claim 15.

17. A host cell comprising the nucleic acid molecule of claim 15 and/or a plasmid or vector comprising the nucleic acid molecule, and a gene encoding a product of interest operably linked to a T7 promoter.

18. (canceled)

19. A method of regulating the relative proportions of two or more cell populations within a co-culture, comprising:
i) engineering a first cell to comprise the nucleic acid molecule of claim 15 and/or a plasmid or vector comprising the nucleic acid molecule, and a growth regulatory gene operably linked to a T7 promoter, wherein said first cell comprises a Split-T7 polymerase which is active within a first temperature range;
ii) engineering a second cell to comprise a growth regulatory gene operably linked to a heat-inducible promoter; and/or
iii) engineering further cells to comprise the nucleic acid molecule and/or the plasmid or vector, and a growth regulatory gene operably linked to a T7 promoter, wherein each of said further cells comprise a Split-T7 polymerase which is active within a fully overlapping, a partially overlapping or non-overlapping temperature range to that of said first cell and/or each other,
wherein raising or lowering the temperature of the co-culture regulates the growth of the respective first, second and/or further cell populations.

20. The method of claim 19, wherein the respective growth regulatory genes are the same.

21. The method of claim 20, wherein the respective growth regulatory genes slow down cellular growth, such as by limiting glucose uptake by expressing a SgrS sRNA which functions to degrade ptsG mRNA that encodes for a glucose transporter, IICB^{Glc}.

22. A kit comprising:
(i) the nucleic acid molecule of claim 15 and/or a plasmid or vector comprising the nucleic acid molecule; and
(ii) a reaction buffer; optionally
(iii) one or more ribonucleoside triphosphates.

23. (canceled)

24. A method of synthesizing an RNA molecule comprising:
(a) combining an isolated nucleic acid molecule of claim 15 and/or Ma plasmid or vector comprising the nucleic acid molecule, with ribonucleoside triphosphates and/or a modified nucleotide and a template DNA molecule comprising a T7 RNA polymerase promoter that is operably linked to a target nucleotide sequence to be transcribed, to produce a reaction mix; and
(b) incubating the reaction mix to transcribe the template DNA molecule into RNA.

25. The method of claim 24, wherein the incubating is done at a temperature of less than 40° C.


SEQUENCE LISTING (27 sequences)

SEQ ID NO: 1 - protein, 883 aa, Artificial Sequence
  T7RNAP
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 371 aa, Artificial Sequence
  Tlpa36
MRPATYEPEQIIEAGLALQAEGRNITGFALRNQVGGGNPTRLRQIWDEYQASQSTVVTELVAELPVEVAEEVKAVSAALSERITQLATELNDKAVRAAERRVAEVTRAAGEQTAQAERELADAAQTVDDLEEKLVELQDRYDSLTLALESERSLRQQHDVEMAQLKERLAAAEENTRQREERYQEQRTVLQDALNAEQAQHINTREDQQKRLEQISAEANARTEELKSERDKVNTLLTRLESQENALASERQQHLATRETLQQRLEQAIADTQARAGEIALERDRVSSLTARLESQEKASSEQLVRMGSEIASLTERCTQLENQRDDARLETMGEKETVAALRGEAEALKRQNQSLMAALSGNKQTGGQNA

SEQ ID NO: 3 - protein, 278 aa, Artificial Sequence
  Tlpa36 coiled coil domain
AVRAAERRVAEVTRAAGEQTAQAERELADAAQTVDDLEEKLVELQDRYDSLTLALESERSLRQQHDVEMAQLKERLAAAEENTRQREERYQEQRTVLQDALNAEQAQHINTREDQQKRLEQISAEANARTEELKSERDKVNTLLTRLESQENALASERQQHLATRETLQQRLEQAIADTQARAGEIALERDRVSSLTARLESQEKASSEQLVRMGSEIASLTERCTQLENQRDDARLETMGEKETVAALRGEAEALKRQNQSLMAALSGNKQTGGQNA

SEQ ID NO: 4 - DNA, 20 bp, Artificial Sequence
  pT7
taatacgactcactataggg

SEQ ID NO: 5 - DNA, 15 bp, Artificial Sequence
  Linker
ggcggttctggaggt

SEQ ID NO: 6 - DNA, 20 bp, Artificial Sequence
  RbsDefault
tttaagaaggagatatacat

SEQ ID NO: 7 - DNA, 81 bp, Artificial Sequence
  gabDP2
gagattttgggctcgtcggggattcgccgggtgctgcaaaaccatctacgctcaggactgggcgagatgaaaaactcgctg

SEQ ID NO: 8 - protein, 627 aa, Artificial Sequence
  Fcs
MNNEARSGSTDPGQRPRYRQVAIGHPQVQVSHVDDVLRMQPVEPLAPLPARLLERLVHWAQVRPDTTFIAARQADGAWRSISYVQMLADVRTIAANLLGLGLSAERPLALLSGNDIEHLQIALGAMYAGIAYCPVSPAYALLSQDFAKLRHVCEVLTPGVVFVSDSQPFQRAFEAVLDDSVGVISVRGQVAGRPHISFDSLLQPGDLAAADAAFAATGPDTIAKFLFTSGSTKLPKAVITTQRMLCANQQMLLQTFPTFAEEPPVLVDWLPWNHTFGGSHNLGIVLYNGGSFYLDAGKPTPQGFAETLRNLREISPTAYLTVPKGWEELVKALEQDPALREVFFARIKLFFFAAAGLSQSVWDRLDRIAEQHCGERIRMMAGLGMTEASPSCTFTTGPLSMAGYVGLPAPGCEVKLVPVGDKLEARFRGPHIMPGYWRSPQQTAEAFDEEGFYCSGDALKLADARQPELGLMFDGRIAEDFKLSSGVFVSVGPLRNRAVLEGSPYVQDIVVTAPDRECLGLLVFPRLPECRRLAGLAEDASDARVLANDTVRSWFADWLERLNRDAQGNASRIEWLSLLAEPPSIDAGEITDKGSINQRAVLQRRAAQVEALYRGEDPDALHAKVRP

SEQ ID NO: 9 - protein, 276 aa, Artificial Sequence
  Ech
MSKYEGRWTTVKVELEAGIAWVTLNRPEKRNAMSPTLNREMVDVLETLEQDADAGVLVLTGAGESWTAGMDLKEYFREVDAGPEILQEKIRREASQWQWKLLRLYAKPTIAMVNGWCFGGGFSPLVACDLAICANEATFGLSEINWGIPPGNLVSKAMADTVGHRQSLYYIMTGKTFDGRKAAEMGLVNDSVPLAELRETTRELALNLLEKNPVVLRAAKNGFKRCRELTWEQNEDYLYAKLDQSRLLDTTGGREQGMKQFLDDKSIKPGLQAYKR

SEQ ID NO: 10 - DNA, 52 bp, Artificial Sequence
  PTlpa
tttaatttgtttgttagttagtttatttgttggtttgtttgtgttataatat

SEQ ID NO: 11 - DNA, 227 bp, Artificial Sequence
  SgrS
gatgaagcaagggggtgccccatgcgtcagttttatcagcactattttaccgcgacagcgaagttgtgctggttgcgttggttaagcgtcccacaacgattaaccatgcttgaaggactgatgcagtgggatgaccgcaattctgaaagttgacttgcctgcatcatgtgtgactgagtattggtgtaaaatcacccgccagcagattatacctgctggtttttttt

SEQ ID NO: 12 - DNA, 423 bp, Artificial Sequence
  Mutated coiled-coil domain of CT7 protein fragment (Mut1)
gcggtccgggctgcagaacgccgggttgcggaagtcacgcgtgctgccggtaaacagaccggtggccagaatgcgtaacaataaacagaccggtggccagaatgcgtaaatcgaggattaccactacgctacgtgtaaaaacgaggacctgatgcactttgacgacttgggatcgacgagagcagcgcgacagaccggtggccagaatgcgtaacaataaacagaccggtggccagaatgcgtaaatcgaggattaccactacgctacgtgactacgctgatgtacagatccatactgcatgttcattgacgatctacaacgcatcacccagctggcgacagaacgcatcacccagctggcgacagaaccagtcactgatggcggcgctttcaggcaataaacagaccggtggccagaatgcg

SEQ ID NO: 13 - DNA, 270 bp, Artificial Sequence
  Mutated coiled-coil domain of CT7 protein fragment (Mut2)
gcggtccgggctgcagaacgccgggttgcggaagtcacgcgtgctgccggtgaacagaccgcacaggcagagcgggagctggccgacgccgcgcagacagtcgacgacctggaagaaaaactggttgaactgcaggacagatatgacagtttgacgctggcgctggagtcagaacgttcactgcgtggtgaggctgaagccctgaagcgtcagaaccagtcactgatggcggcgctttcaggcaataaacagaccggtggccagaatgcg

SEQ ID NO: 14 - DNA, 61 bp, Artificial Sequence
  X1
gcggtccgggctgcagaacgccgggttgcggaagtcacgcgtgctgccggtgaacagaccg

SEQ ID NO: 15 - DNA, 125 bp, Artificial Sequence
  X2
cacaggcagagcgggagctggccgacgccgcgcagacagtcgacgacctggaagaaaaactggttgaactgcaggacagatatgacagtttgacgctggcgctggagtcagaacgttcactgcgt

SEQ ID NO: 16 - DNA, 27 bp, Artificial Sequence
  X3
cactgcgtggtgaggctgaagccctga

SEQ ID NO: 17 - DNA, 64 bp, Artificial Sequence
  X4
gcgtcagaaccagtcactgatggcggcgctttcaggcaataaacagaccggtggccagaatgcg

SEQ ID NO: 18 - DNA, 28 bp, Artificial Sequence
  X5
caataaacagaccggtggccagaatgcg

SEQ ID NO: 19 - DNA, 64 bp, Artificial Sequence; identical to SEQ ID NO: 17
  Tlpa positions 1050-1113

SEQ ID NO: 20 - DNA, 64 bp, Artificial Sequence
  Mut1 positions 360-423
gcgacagaaccagtcactgatggcggcgctttcaggcaataaacagaccggtggccagaatgcg

SEQ ID NO: 21 - DNA, 61 bp, Artificial Sequence; identical to SEQ ID NO: 14
  Tlpa positions 280-340

SEQ ID NO: 22 - DNA, 61 bp, Artificial Sequence
  Mut1 positions 1-61
gcggtccgggctgcagaacgccgggttgcggaagtcacgcgtgctgccggtaaacagaccg

SEQ ID NO: 23 - DNA, 28 bp, Artificial Sequence
  Tlpa positions 1086-1113
caataaacagaccggtggccagaatgcg

SEQ ID NO: 24 - DNA, 186 bp, Artificial Sequence
  Tlpa positions 280-465
gcggtccgggctgcagaacgccgggttgcggaagtcacgcgtgctgccggtgaacagaccgcacaggcagagcgggagctggccgacgccgcgcagacagtcgacgacctggaagaaaaactggttgaactgcaggacagatatgacagtttgacgctggcgctggagtcagaacgttcactgcgt

SEQ ID NO: 25 - DNA, 92 bp, Artificial Sequence
  Tlpa positions 1022-1113
cactgcgtggtgaggctgaagccctgaagcgtcagaaccagtcactgatggcggcgctttcaggcaataaacagaccggtggccagaatgcg

SEQ ID NO: 26 - DNA, 125 bp, Artificial Sequence; identical to SEQ ID NO: 15
  Mut1 positions 341-465

SEQ ID NO: 27 - DNA, 28 bp, Artificial Sequence
  Mut1 positions 1022-1049
cactgcgtggtgaggctgaagccctgaa